SEQUENCE LISTING (64 sequences)

SEQ ID NO: 1 - DNA, 57522 bp, Homo sapiens
gaagcacgcccacaccatcccccctcactccaccttggggtcttcatgtctatttccaaagggcaggagttgggactcattgctgtcataaacgattctccccaatctgctggctcaaggggctccccagagctgaccgctggggctggggcaggggcacttgagctgaggctcagctgctttgggggacctagctgcaggtgggtggaacttatagctcatctgtagttatagaaagagggagacaaatgcgggccaggattagaaagtagacctgagtcacctgatccagagaccaaagaagtttggcaaagggagaaaacaaaactgccacctctcccacaaacacagaggaccgtgggaagatgggcagtggaatggtcaagactcaaaggatagaggccgggctcagtggctcatgcctaatcccagcactttgggaggctgacacagaaggatcacttgaggccaggagttcaagaccagcctgggcaacatagtgagactctgtctctaccaaaaaaattgaaattcaaccaggcatggtggcacacacctatagtcctagctactcaggtaacggaggcaccaggatcacttgagcccaggaagttgaggctgcagtgaactatgattatgcgactgcactccagcctgtgtcacagagcaagaccccgactcaaaaaaagaaaaaaaaaggctcaaaggatgagttcagggctctgcttctcaaccaaaccttccttgcaacttccaccaggtggcctagtttttgtcttgcctgcattcttgaggctccgctcagggtatggagaggagcctctgacagggttgagggccttcgcaggccagtcagggaaaagagaactccattcttctaaacgtccatctttctacttttacctgccagaagggagagctcagctctctcctgaaagagccctggtgcccacctctcctaaaaaaaaaaaaaaaaaaaaaaaaaaaaaaaaaaaaaagagtggactatagacccccaagagattaaaaagaatagaagggttttatgaacttgcacacctcagacacataccaggaatctgcttgttcctttctgaacaactccagagacccctcctgtaacctacagagctccctgccagcctcagatccagatagacatgagctcctgctcaatgaagccccctcccaagacctcccttacctggcctcccacgactgtagccttgtcccccatggcaagggtcgcctcctggtggctgctgctggaggaaacacgagtctggcatggatggggggctaggatgggttatgcctccccgcagtcccctggcctatcccaccctaagtgctaaagccctggggtcgctgatgccagaaatacggatgggagctgtgggtggggccgccacagctcacacctactcacctgggttgcccggctggctctggctgtggctctgcactgccccacagaacgggtgctgcggctggggagggggattattctccactcaaattgtgcttgtctttactggggcctcccaccacctggcccccactgctagaaagcctctcccacgccactgagcattcccccattctccctccctgcagccttctcaaatcctttctctcccttcctgatgacccttctccagccaggatctccctgtttactctcagtctttggcccccagctctctctagttccccacctcgggtctcttccacccagcctaactttggactctttcccccgcggtcataggcctgcctctgttggcttcggggtccctcctcccctgactgctcctcctgggtgtccctctgctctccctcaaacctctctttcctccctctctccaacctgcgcgggcccattcctcattcctttaacgtttctcattcctcgagcccccgacctccctgagtccacaaccctctcaccgaggcgctgcacccgcagggactcggctgcctgctcaccccagggcagccagacacaaagcagccagcagagcgcagacgccaggactcccatagggacacgaggggaccggaggacttgagcgcagggccagcctcccgaggtgcctccccgggctaaggcagggtcacacctccactccgcagccgaggtccctcttgttctcatgccccagggctccctcagcccctcctcccaggctctcagctcctccccgaactagagtgacaggagtacccagcttattaccataatttaggcgcctgtccatagcctagcctctgcttttcttggcctgtggccacacctccccaggggaggctggattcagattactcagccctaaattgtctagggaagcatagaggcagcctatgcaacctccagctccccttacctgggtcctggaagcccaataccagagacgcaagatgcaggtatgagtctggccctatcctctccttttacagagggccatgctgaggccctgggacatgccactcagggatcagtggtcaataacagagcctgcagagtcctagcctatgttctcaccatgccccacctcagcttccacctgggtctccacatccgtatcctatgaccagttttgagcacctctagggaatggatcatgccttagtcctctgaatgcccagcaccagctcctgcccgagctcaggaaatgacgattgaataataaaagtgtttcatactcctatgttattgccaccatcaaggccaaactttgacaaaacaactttatatgtgtgaacgaggtccactgtctgcccaggaggaattttaaaatggcaaaaacatgggactgagcacctggggaatctgtacatcattcacttcgtcatcacttagggaacaccaactagatgctaggtcttacagaggatgatgatgatggtggtggtggtggtcgtggtggtattggccccagtagacttctaggcaagggtatagatttgagatgcattcattcattcattcattcattcattcactcaacaaatttgtttggcattgattccatgccatgcactgttctaggcatatagtggtaaataaagcaaacacagctatcttcacacaggaggaagctgggggcacagataggaagatgaagaggaaagagctaaggatggagccttaaggaatgctggttaaggaacaggcaaagactgattgtctccaaggaagcttgaaacaaaaattcatgagagaggagaaccaggtctcaatgctattctcaaaacaaagcagggggcattttaaggtggagggagtctgtgggggtaatggccaatgtaaaataaggattggtgtggcaggtgaacagcattcccctggataaggacattcattcacctctgccaggattctaaatacaaaataaacagccatatgacattcttttccaatgtagagaatcaatgaagtaagtcggaggggaatgcattttgtgtggccttttctggaccattcaaaaatcccacaagtctttccattttatcgctgacttaatagttaatgctaaggtgagaaaacagatcatattttaaggttcttctccaagagggatttacatcttaaactaggcaacaaaaatagtgtgcttgtaaaacttcagctattggggagagggacgcagaggggtgtttaggtgccccagagttacaattgtaagtggttacctgatgtaagctgctgtccatctccttgcccctgcccttgacagggcacaaagggctcaggtgcctccctgggttaagttcaggcacaagccccatacttagcctctgatttacacatttttatcacccctctcctagcccctctttcctttcatctctttccaggcctggttcatcattgtctttgatgaatctttcctttcattcctttccaggcctggttcatcattgtctttgcaccaagcactagacctaggagttaagcaaatgctgaccaaattgaattaaatgaaccagagggtctgggccctgggtgaaggtcagccatagatcaaaatgtcactgccaggctctgccaggcttcgatggatatgtgagtgtgttgggtttgggttttgttttgtcttgttttgttgggggtggaggtgttagggaaggtgggaggggaaatcagttgaggggggcactctcacacacactgattcagacccctgcggcccacagtaaaacccagccctgtccccaaggaattcacagaacaaggattgatttctccctggtggaaaagtgcaggaggaagccaggactgaaggtacgctggaggtgagggcgtagcaagggctgacagtccaggagggccatgaacctcgacaagagtatccagagaaggccgggcgcggtggcccgcgcctgtaatcccagcactttgggaggccgaggtgggcagatcacgaggtcaggagatcgagaccttcctggcgaacacggtgaaaccccgtctctactaaaaatacaaaaaaaaaaaattagccgggcgtggtggcgggcgcctgtagtcccagctactcgggaggctgaggcaggagaatggcgtgaacccgggaggcggagcttgcagtgagccaagatcatgccactgcactgtagcctgggcgacagagcgagactccgtctcgggggggaaaaaaaaagaatatccagagaaaacggactagattgccccgccccccgcccgtgtaaatagtttccgtatctctctattccggtccccacaaaaaagtcccaaacctcctccctacgtctccacgatcttcttcctcaaacgcatgtgttcaggtaccacttccagagaaataccagcttgaagcccagctactgccacctcaggcccataggcacactggggcccatttgctcccaggcttcagtgggaggcgacgactcagcaccttcgactccagcctcgcagcggccccgccccacagaggcctggccccgcccctccgcgctcaggccccgcccccagctccgagggcggctggcccggtcgcggtcgcggctctttccagctcctggcagccgggcacccgaaggaacgggtcgtgcaacgacgcagctggacctggcccagccatggaccgaaaagtggcccgagaattccggcataaggtcagagctgcagggcgccccaggcttctgggactccggagtcctgggcgcggtgggtagggggtggacaccccggcactgcccctcccttttccggccccacctgatggctctggttgggctgggacacccgagggtcgtctggctggcagcagggatccccagtaaagtgagggagggaatgcggggactcccggctcaaggactgctaaacgagtcgatcttctgccagcctttctccctctgccttccaggggcagggacgtctctggggtttgaattccttccagtcttggccgcttttctgaggtgccccctttgtgggcaagcccctctccttcctagtgcccccagctcagggctgttgaggcatgtggagacagtctggggcagtatctgagaggtgagggttgggagaagggaaactagacgtctctctctctgccttttgacctcagaacatgagttagaagcatttcagccctgcctgcctaagggcgtttcttagggtctgagaagtagctgaggagctggtgctgacccgggtgcggtggggaggaagggaggaggtactgagggcgtcggagctgggctctggccggccagatccttcaggcagagccgggtccaccctggtgtgtcccagtgaggggcctcactggtggtctgggattctcaaggaccatctctggaagtggccaggtttcacacagggcatttcgagaatgattcacaaagctgtgcagaatctgctgaggccctgagaccagagagggccatcaaagagatccagcctacttccccaccaagtcttaggatgcctctgagtctggggatgaagccgcttggggggggtgggtggtatctggagctccccacaggccctgcctgaggggcaggtaactattgataacttaactattgataactgtgtgacccaaggctctcccaaagaccccaggggcagtgtctttagaccaagccaaacctcttcctggtttgagagctgagctcaggtagggcaggtcagggtgcttaactccttccttcccataaagaggcccataggagcccaaggagctagccagtgtaggggccacaggggcttggggccaagggcccggggtcagttattcatttaataagcatgttttgagcatcctctcagccatacccagtctgtgccctttgctggtggtgtaggtgggaggcagaccctaccctggtaattacccatgtctgagtgctgcgtgttgaggccaagcttttgtaggggcacaaagggacagctcaaactggcagaaggctcctgaaaacaaggtcttgggcatttctggtcctgctcccaggggtgggtgatagctggaagttcagcaggaatttaggggctggcgaataccaaggggagcttgagagagcattcactgttacatcctgttgcaaagagacatgtcggaagaaatttcagccactaaggacattttgtgagtgtagatttcaggcaacccagtttgaaggagctcagccttccatcccccaacccaagactcagggttgaatttcagtttcctgcccctggcctgaaataacatcaggtcttctgccttcactctgtggggtacctgctctctcttttttgtgagaatcatctccagggtccctggtgtctgatgcagatcctgggtttgccctgttcctcttccccaggtcccacaggctccaaagggcctcagacctccacgtttcctctgccctactcttcctcatcgcaacagtcattacttattaatatccctgtgtgcaggcagggcaggcgccatgcgctaggcaaggcccaggctggactttgtgccctgctgccttggagacagccacagccttcccctcccaggttggggatcatccaggaactggggagagaggatgaagcacagaggatagaaaggaggcacacagacatgctgggagaaatttacagcttgctgttgtttgctttgtaggggtcccttccttagtgttttggaagaaatggtttgtgatctaaatccttagttgttggaagtaatatttaaggtagtgtgttataatggaaagtgctctgtgaccttgagcaagttacttaatctctctgtgcctcagtgctcttacttgtgaaaagagataacaatatttaacgaataaggttaccatgaatgctgaatgagacctacatgtgtgtggggagcttaaatagaccctggcacttagggagctctcaagaaatgtccatgttgattattatctggagttagcagaactgggaccaaatcttagccctgtcactgtcaggaagaccttgaccaagccacccactctccctgagttttagtttcttcagctatgagatggagtttgtagaacttacctcacaagacagtcaaaactaactagcaattaaagtactggctgctctgggcccacagtaaccacactgtttccagctgatacctgcagagtgtccagtgggagccaacaggctccaggcatcgactccctttgatttgtgaagttaccccaaggccgccaggagcacttgcatacccttccagtaactagtagcaaccttgggccaggatgtgggggagcagggcttggtcagagactgttttctccctcagagaaccagctttcaaagggagactgctcttctgttcgcagcaccagcacagggtaggaacttggttactcattggcttaaaagtatttattgcctcttcagcaatcatgcatttatgagcatagctgtgtattctgcctgaggccaggcatctgacaaccggttgggatgaataagatcagaaagagccaccactctctaggaatttgttaatcattcatttattcacataggcaataaatattgactgagcccttaatatatgccaggcagtactctaagcatcaaaaaacaaaataaagcccttgctgtcttagacagtacattccagtagggaagacaggtagttaaaaggaaatcaacaaaagtatcatacaatgccagatagtgataaattctatgaaaaaataaactaagatggggagagagagatagtaagtgacaaggtaggcagtcacgaaggcctctctgaagaggtgacatctgagcagagacctagaagaagtgaggagaaggagccacagagatacatgtagaagagcattccctaaagtggaagcaacaagtgtgaaggccctgaggcaagcagatgcccgcctgtatggaacagcaagtaggacagtgtgtcccagaggaatcgtgagtggagaatggtgagaaatgggtcggaggggtggtaggggcctggtaggccatggtcaggtcaggattttcttgtaagtgtgaatgatttacatttaaaaggaattgttctggctgctctgtagagaatccactgaggggcccaagagtagaaggggacctcagtcaggaggctcctgcaggagcccaggccagaggcaggggctcggactcgggtgagaatgggtcggattcaggatacatgttaaaggaaaaactgacaggctttgctgatggattggctgcgagcgtagaaagagaggcatcaagggtgaatccacatttggtggagacggagcaggttggtggagacggagcaggaggtggtggaaacctagcgttccacttcgaatgctgtaagtttgagacgcctgctagaggtgactgagaaggctgtaggtctggcctggagataagcatcggtaggtccttgggggtgtgagtggtatttaaacccctgagatgaatgaggtcacttagagagacagtgcagatggagaggagacctaggacagagcccggggtacctcaacttttggaggagaaggagcagcaagcgaggaaggaaagcaaggagaagcagggcgtgattgctgtgccaggcacagggtgaaatactacaaactagctgacatgtcaagagcctctgaaaagatgaagggcactgtctatgtccttgatggtggtgatgctttcacacgtgcacatttatccccaaactcatcaggttgtatacactaaatatatacagcgctttacatgtcaggcatacctcaataaagtggttccagaaaaagaaaagaagtcaggtgtggcggctcacgcctgaaatcccagcattttgggaggctgaggtgggagaatcacttgagtccatgagtttgagaccagcctgggcaacatagcgagaccccatctctacaaaaaatacaaaaattagccaggtgtggtgttgtgcacctgtagtcccagctacttgagaggttgaggcaggagaatcaattgagcctggaggttgaggctgcagtgagctgtggtcacaccactgcactccagcttgggtgacagagtaagacctggtctcaaaaaaaaaaaaaaaaagaaaaagaaagaaacagaaacagaaagaaaagaaagagagagagacagagacagagacagagagaaccctagacaagaaagaaagaaagcaaaagaaaaagaaaagatggatgataagaaaatgagagtcaaataaagcctggtaccactgggatgcacactctaaaggcctgggaagaagtgtggctggatctattcatcccactaacatctacagagggccactcgctgcccactgctgtggatatagaatttatgtccacttattgtcacttagttttatgtaacaaacacaggacttactacctgccaggcactgttctgaactcttcataattattaactcattaaattaatactaaaaaacaatgattaatctctcatagtgattaaatcccattttaaaaagagatgttagtcctcattttacagataaggaaactgaggcacagagaggagcagacccagttggggaaggggttctttggctctaccaccacactcacaagccaggcctgtgtctgggccacacacaggcttgtggggagacaggagggtaaagggagaaagttcagcacagcttggtgagtcccatggcagagttggggacaaagtgctgttgtgtgcacagagaaagatgtggccagctttgtgtgggagcctaaggaaagacttggccaagaagaggcgacatttgaagtgagtcttaaagatagaggaggagtccacagagaggaaatactgctggtaccaccattgctaatggctaaccagggtggcagcagggagcaggtggctcgtccaagggggaagccaagaaagctttatgaacaggttctacagaaaagggcaggattaaatgacccaacaagctgcaccctggggccagatgcaggaggccagcatccctgaaggggccagtagagggaaggttaccagaacaggtgagaaccagggctgccaaagaggcccagacagcagctgcagccttgggtggaggaacctgcctaactgtggcccagcagccagcccccagggactaggagcctcagttcctgtctccccacgcctctcatctcctgcttgtgcctcccgatggctgaacacagcagaaagccagaggggagaggagcccaggcagagccctctggacaaagggcagggtggagaaggctggaggcatgaaaggaaaagatctagcacacattttggagaccttgaaatgtcccaggcattgtcatacgtgctttacacatactcactcatttaatccccgcaacagcccaaagagacttcatcaagcagaacaacatgcattatttaatttgttctggctctctttctccctgtttggctgggtgcacacctaaagttgaatcttcctgagttgactgtcccatggttcccctgtgtagctatcctgaagggccagtccatatgggggaatacagagggatgagactggagggtaccacatggccaaacccagcttttgcctccaataccctagacaaggggcctgaagattgtgagggtggagatgctccctgtcccctcctccctcccacacagaccaatagcacagtgccagagaaacatcagtcagcaaatgctaatctaggcagggctggcagcaggggcagggggtagcagggatgataatagagatccccaacagctatttgtagatggtgggctcctttagggcttctgtgcttaatatcaagagggatccaagaaaaggaaaggctttctaaatctagtcggagaaagaagactggtgtctttcccacagtaggtgttcaatagatgtgtaatggacaagtggacaacaaaggagttatatttcataagtggataccatgtggtagattgaaaaaaagcagaggttttggagccagtaggcataggttggggtctcaattctgtcacttctctgagcctctgtttcctcatctgtaaagtggggatgataacgttcacctcagagggttgttaggatattaaagataatacacgtaaagttcctcaggcagtggacagtcagtagggagaggctggctgggataagtgagccagacagaaagagactcaggctgggaggcaggtgaggaggctccagactctagaagaggggacttgggcctcatctgaaatgaaggcaggattcagatgagaggaggaaagctgtccattgtggatagatggagtcgctgggacctacttttttgtgatgaattggaagtgaactaaggggaggcagacccagaatatatgtgctgaggaccagtggaaaggtggtgacccaggcctgggccaacaggtcagaaagaaggctctagactagagcaaatagagttcacgtttcatcaacggacgccactgggcaccgtgcgcttgtgtgcatgacatggttctgggttccacagggaaatgaagaacatgtttggaaggaagggaagaaaggagtgtgggagatttactgcgtgcctagtgcttcgtatgtacctgagtacagggtactgggacaatggtacaaagcaccctagagcaggggctccccaaaactgatcctcgggctagtgctaggcagaattccagaagagaggaaactatataattttttaatattggaaaagtaatttgatttggaccacagggaagactacaaagaaaaagtaatttaagtgatggtggtattgttactgcacgttcaggctttagagaaacttccatctttctcagctttctttcctggtgccttttaatgcctgaagagtgaggtgtgagtgtgtgttttcactcaggtgtggtcagagaacaaagcagtgctgttctttctgagtctttctgagatatttctgggtgagaatgatccctccctttgcaggatctcctgtgtaaccagttttcaagtttttgatgatctatcacttagattcatatttaaagagcattctacacaaaccagatctattttccctgttagctggtatggtctatagagaattgtttaaatagacaagtcagacatggcggtagatggaatgttctgagtgaggacaaggagattccagtgtgtcaggggaaggatctgttccactgcagctgagtcccacttgggatgtggtgaagcgagcaatggcagaactgaggacagggtttgagtgacctaaccggtgacagtgggtggacatgaggccgaagagctgagctctgcagctgtctcaggagacaggtaggatgagacctctgggagcagtggtcagtgctggagggctgctgacaagggccaggagcccgggaccttcagggacaggctcctttccaccaagaccatctccaagtgatctgtgcttggcccagggaagggagaaaaacagaaccctagaccctaacattgcaagttaccttactcttctacctcagttttccacctaatgcacaataaacatggtctaaggaggacagttcctcactactgaaatctaatgctacagcaagatacatttctgcaaagagggataagagggaacttcagtcctaaggcctcagtcaataagagattctctgtcccatcttctttcttgtgtcaccacccagggttataactaggctagaagtctttagtcagggtgtcctctcttcagccaaagcagacgtgatttttatgctccccttagaaagtacaacacttgggttcaaagagtcattcaaaagatgtcccattttctcactcattatagaccaagccaaaagtgttttcttaacagtgcagaggagagagatggggcttagagataagaaaggagttcttgaaagcaaagggttggaaattttggcctaaagggacattgggagttattttcccctgccaggcctgagtcacaatcaatggtcatcgtggcgtagcagaaagaacatgggctttggagtcagacttaggttcatatcctagctctgcttattagctgtgggacactgggtgagttgacttaacctctctgatcctcagtttcctcagctgcagcattatgtgagaatattgccccaatgtgataaacaaatggaataaagcccatgaaaagctcctggtgccaccgcatggggcattatggggacaacatcatttcccttccccttctgttcccatggttacctccctcccacctgaaccatgtgggcataccaggaggcaggcagataaattcattcaatacttctttattgagagcttattatgtgttgggcacgagaaatttagaacaaaatagatctcatctttcccctcatgggatttttctgtccagcgaaggtgacagagaaaacaattcacagagaaaacaaaccttaaattacaaattgtagtcgatctgtgaaggaattgaaacatctccggggtgcaggagtcggttctgtctgggtaggtgagcagggaagacctctctggaaaggaggtggccaggcagggaagtgggggaagcagtccaagcagagggaacaagcatacgccaaggccctgaggctggagagcgttcggcctgtgggaagaactgaaaggaggactttgtagccaggaagactggtaggagaggagattgggctttgataagtcaaagtaaggagtttggatttaggtttggtttggggtacaagaaactactgagcaagcaagcaacatatctaatttataaagatatttctcgcccctgctgctaggagagttcatactcctccatcacagcccagtgtgggcagcccaggcctgtctcagggaggcacccctgccccacaggcctgagcagagggggtgagagaatccaggctatgtggagagatgagctttcagaggtggtgggtgcgaaaggccagcctcccaccctaagatttagtaccacccactcaagcagatgcttccatctcctgtcatctgggagctcctttttttttttttttttttgagatggagtctcgctctgtcacccagcctggagtgcagtggtgcaatcttggctcactgcaacctccgcctcccgagttcaagtgattctcctgcctcagcctcctaagtagctgggattacaggggcataccactacgcccagctaatttttgtattttaatagagacagggttttgccatgttagcgaggctggtctcaaactcctgatctcaggtgatctgcccacctcggcctcccaaagtgctgggattataggcgtgagccaccgcacccagccctagctgggaactccttgcactgaggtagggagaaagcaagggtgcccttttggagcaggtgggctgaacttctgtagcaactaaagcccaagctgtgagtcaagcctcccaagttattctcacctttaatgaaatgctcagtctgattttatagggaaggaggtactgtcagatctaggccagaaatctgcattctgtaccccctgctcaggccagaaatcccaagggctgggcccagcatgtcccctctgtggtgggacggacagactgccccggtcttccagaacccttgggatacccacagaaagaggtaacgctgctctggccctcttctgaggacgagtcagtggagagcatgcagcttccagctgcagcctctctatgaagggctgaggccctgggccgggaggctggaggagagagggacccagtgaccccccaagcttccaccttgctctgttacccgttcttgggctgaagagagacccaaaaatacagtgtagagattcacactgaggtaactcagggagtggaattcagggcctcccgctgggattgaggtgctaatgacacaactcctgaacctgaccttagagtgccagccattgacgtcaacaaagttgaaatgatgtaacctgacgctccccctgcggggcttgtgcaggggcctggggagggggaaggagtggccatgaaactgactagtggacagaacccagctaaggtcaggacaagacagagtgaaggtcccctggcactgatgttacagaagaattcggtggtaaggggcttctggagagtggcatgtgctatctaagcgagtggcccaaatccttcctgaaagcatttatccggcactacagccaccatcaggtaagacagtgggcttcttctggccatggatgacacagccatgggggtgagcagcagcactgccatggcagcgtgtcactgtcacatggggattcacatatgtacctatgtgtgttcatccccgtgtgtgcacatattgccccacctggggacaaagggtgcctggccacatctggaggggcagcggtactcctgtggccacgttggggtggtctgcataggtctgatgcattggggtcagaggggcagcctggcctgtggctcctcttctctcctcacaactccagccctgaaaagctgctggggaggcccttggggatgacctctcctccctgaggtctgctatgggggcgggtgctgagcctggagctgtgattctgctattggattttccaggtggattttctgattgaaaatgatgcagagaaggactatctctatgatgtgctgcgaatgtaccaccagtaagtgtgctgggtccagctcttgtgggccacttgggttcctttgtcttcagggagccctgggatgggttgttctgagacagaggagctcagagggtggatgctcacggctcctggaaatcaaatggacataccattcactcatttcagcaactatttacacaagtactttgtacttggctttgtactaggggctgggtatagttgtgagccagacagattggtctctgttttcaggttgctcacagtctgatggaggaggctgtctagtagccagatagattctatagagcatgattgttgggacagaacaagaaatgccagctggccacagcccttgcatcagatgtctccgatcacccacttgctttttgattcattttttctactttataagctcctgccactgctgggcactgtgcagaatctggaaatgaattagatccaatttctttccttgagtaacttgtggtctggtgaggggagatgaacatacactgcaaacacaaagaactctaatataagttacatcaaataactgctaagtagaggtaaaagcagaaatgtgaagaaaggagttttcctttccaactgcgagggaagaggaaggaccaggaaggcttgagcaaggctttgaaggataagaaagatttggggccaggcaaggtggctcatgcctgtaatcccagaactttgagaagctgaggcaggaggattgcttgagcctaagagttagagaccagcctgggcaacatggtgaaatcccatctctacaaaaaaatacaaaaaattagccgggtatggtggcgcgtgcctgtagtcccagctacttaggagactgaggtgggaatatcacctgaacccaggaggtcaaggctgcagtgagccatgattgcatcaatgcactccagcctgggcaagacagcaagaccctgtctcaaaaaaataataataaaagaaaaagattttggtaggtggaatatctgggaagggcattccagaatgagggatcagcatcagccaaagtgtggaggcatgaaagcaagggtgtgaatggagataagtaatctgggggagtaggacttgggagggcacggagatcataaatagccacaaggctggagaagctccatggggacaggtcatggagggccttgagcctgctgagaagagtggactttgtcctctgggcagtaaggggccatcaaagggttttaagccagggagtgccttacactgagaaaagatgacgtgacagtgagtacatgggcaggcagctgcagtcgaaggtctgaacagcatgagggaggaggcatgtgaactgtggtgaggtgaaattgacagagcttagcagcagatacgagtggagatgatgagtgtgtgaggaattgtcagcatctcacacagagctttcccctctggaaagatcccaacagccgagataggcagcgctgagtttgaaatcctggcttcatctcatctgcaaaatgagtcaacaatccctagtagactggtttcctggggatatttatataagagaacaaagtcttctcaggcactggcccggtgtgaagagctctgggcttttcaggagtggtctactccattcctaagcctgggcccagtggctgaaatggcttcctcttggaatccctgggtgcctgaggtcatggccaggggtgaggctccaggcattcccggcatctccacaggaccatggacgtggccgtgctcgtgggagacctgaagctggtcatcaatgaacccagccgtctgcctctgtttgatgccattcggccgctgatcccactgaagcaccaggtggaatatgatcagctgaccccccggcgctccaggtgcagaggaagccaccaggctggaggcagggggtggagagatcaccctgggcggggcagtgctggcagccaagctgcaccatcaccgacctctcctgtgtggcaggaagctgaaggaggtgcgtctggaccgtctgcaccccgaaggcctcggcctgagtgtgcgtggtggcctggagtttggctgtgggctcttcatctcccacctcatcaaaggcggtcaggcagacagcgtcgggctccaggtgagcaaacagagtccgggggagggggagcgagggcctcggacctcctgcctccccctcattcatccactaggctgtgtggcacaacatggtcacccacttttctgagccttcgggtgaagaagaggctggcgcatcctgatgggtgttcttaggctcatagaaatcaggccgcaggcaattgcctgttttcttgagtgaagctggtaacctggctgctgcctgcttccaactgctgcctccttccagctgctgccgctgcacttccccccacctcccctactccccaagagaggaagacagtgatgctggcatatgaagttttggacctgttgccttttaccagcagggggaaagaaagcctggtgcagtgtgatgccgaagagcatagactctgaagcagggttagccgggttcaattctggctttgctgttcattaggctgtgtgacttggtggaatgacttaaccctgtgcttcaatttcctcatctataaaatgagttgccgatagtactgtctacctcgtcaggttttgttagtaaatgaattaatagtagaaagtgcttatagcagggcctggcatacaaatgctgtgagcctggtaagtgaacagagagagggagatttaagaaacgcctggaatgtgccaggtcacatgctcacaagcagtccttgctatatgcattgaacggatcgtgcccattttacagaattaatagaggctcagaggccagtaagtggcagagccaggattagaaactaactgggtctcctgactgccaagcccagaaatctctcttcagcaacgcaggtgcctctcctttggggtccccacacctcagggcctgagcagagatgggcagacctccaggtctcactcctacctgagcccagggctgtgtttttgtgtgttgagataaaggaggccctcccaccatcaccaagagcttccagcgggtttgttatcaacatcccaatccaggctgccaagcttggggctttcaaggggctcgaaggctaatggtacaagacactgtggcgtaaggggtggaaacagggagctgacagacaccgctttgttctaaatccctgtctcacggctccctggtggtgtctgaaatttcagccccttcattatttctttcctctgcagcacattttccagctcagaaatgcagccagagaaaacacataatgagcgcctctcttggcgtcagctgaggccgccttttttccagggcgagctctcttaggacaagcagttctcaatgctgcctcgatgactgggggcgttggggtattttaatgagacctacagttttaccttcctggctgtttctcaggcttatgaattatcggccctttctctagctgacgggttcatctctcctttgtgccgctgtccctcagatcgttatatcatcgtggcccttgcacaaagggccctttgcagggctccacacagggcgagacggggaggaaagttgatcctgcaacctgagccaggggctgtgtgggaatcattccgactggggttctgggcaaattccctttaggaataagacagggaactttactcagaggagcttcgggaaaaatggctgcatccattgacctgtctggggttcatgcttctggggagatctcatgcctgagggcaactggaagaagatgctggaaggcaggggatgagcaggttcagatacagcccggctgggctaaagacctgtgctgatttgacctgtgaggctgggtccccagtggtgggcttggaccctcccacaggacctagtcctgggggtccacccctctgcccttgtcccctgctggagatacttggtttttgtttttttttccccaagaatatcctaacttaacctacatcctctgccttgcacagggcagcctgtgacatacaacttgctgtatattccagacctagaaaattattctgtgtgctttggttttccctgtcataacatggacagctgcctttgtgtgggacttgagggctctgacaggtggcaaggatccagagagggcaggatgcagggaattgcagctaggcttggccggatgcccttcttttctacttccagacacccaagagacaccacttgtcgatcagggagacctgacttcaaatcccacgacactgtttactattggggtaaccttgagcaagtcactttacctctctgagcctcagttttctcatccgattaacagagatacaaattcctgctctgcagggttgttgtgaaaaataggtggaaggagttagtctggccgctgtccttgaattacatgttcccagaaacctagagagttctttagtgggcccccaccccagtgccattttgagcccttggccactcctgtcaggtccctgagaagactggggtctgtgtcccggagtgggagggaagcgttccttggaatagtgagaaggtgactctgtgggaatgctgtagagggcaggagttgccctagaggacccctcggaggctgcatgtccacccagcccctacctacctagacccacagggagtccagcttgcatccctcacgtgtgccagcacgtctccaaagggtgagcacgtgtgtttcgagttaagcccccagctgacctgcactggcctcagaccggaacctctccaggagccagtctctgttttgcagctactggctgtgtgaccttggacaaaacctcacttccttgggcttcagcttcagctgttatctgagagttcctcctgccctgtggttattaaatgaggagctccagaattgatccccagggccggggtgcctggaggagccggcagtatccagcagggggcaatctcaccacggctctgtatccagggctggctgcccagggcccatctcaacaatccactgtggcctaagccctgagaagagagatctgagctgagtattcagggatcaggactaactcatgataacaatagcaatcatgtattgagtgcttactgtgtggcaggcactagctgtctttacatgcacaagttcacttaattctcacagcaaccttggtattccccattttacagacgaggaaaacaggttcagacagttcaagagacttgctcaaggtcatatagctaataataataaaagaagggatttgaacccagcacatctgatgccaaagccctgtgctcgttcactgttctttgcttgctcccaaaataggaattcagaggtcagggccacagcagagttaaaatgttcatcaagtttccatatgatgggaaaaaaaaatcatatgtgtgtgtgtgttggtgatgtgagcttgggtcaggagtcacagaaggtccccaccccgactcagttacagtgttgtagcaattaacagagatagggagccaacttcctaggggtgggttgggacaaagtcccggtaagaatagcttaaagctgagtgaaatgtcaccctttgcatagaatccagaatctgatggtgccccagtggagtgaaaggggcactagagtgagggtgcagagagttctgagatcttctcccagctctgctgcacacccgctgtgcccctcacccctgtcttggtttctccatctgtaaaatggggcgacaagactgcttggacatgccacctgaacctgggatcccccgggctgatggagggtgggttggttgagatcaaggcttattccagggggtggcacagccacccttcccttttccggagagcagtccgggagcatctggtggtgagtctgccccactgcctgatgctccctccacctggtgctccctgcctctctctgtggtcaaggtaggggacgagatcgtccggatcaatggatattccatctcctcctgtacccatgaggaggtcatcaacctcattcgaaccaagaaaactgtgtccatcaaagtgagacgtgagtgaggccagagcagggcagtactccataacggtgggagggagggagggcgggggagcagggcagtactccatgacggtgggagggagggagggcgggggagcaggtcagtactccatgacggtgggagggagggagggcgggggagctgtcctaacccctgtgcctttctcccgcagacatcggcctgatccccgtgaaaaggtgagaggcccctcctctgcaggccaactcttccctgtgggcccaggatcctggtacagccctggggtccggctcccaccatgccagccctgcttctgggccagtggaggctggaggctctagacatggtggatctggatgtggggcctggttcctcaaacgtctctcgctaaccaccctcccatctattttcccttcccatcagctctcctgatgagcccctcacttggcagtatgtggatcagtttgtgtcggaatctggggtaagggccagacctcctgtgatggggtttgggtggggtcatcttcaaggaggggtggccggtcctgaagggagggcttgctctagagatgcaccctcaggggcttcacacaggctcccagggcagccagcacaccgctgtggggcagcagccctcggccaggcccagctggtgcagacacatccccagggacggaatgatgatctggctggcgtgagttcagcagtgctcgccctgcagatcccacaagctcaagaggccgcttgcacgcatgtggacactccgtgattctgcttctatctctctttcagggcgtgcgaggcagcctgggctcccctggaaatcgggaaaacaaggagaagaaggtcttcatcagcctggtaggctcccgaggccttggctgcaggtgggtggcaggcatgccctggggtcattcgtggccagtgcaccccagcaggcccctattgccctccccttcctcactgccacttccgaggaaaccttgcccaccaggggtgtgactgtccatgggtgatgatactttttttgttagatacagggtctgactctgttgcccaggctggagtgcagtggcatgatcatagctcactgtagcctcaacctccccagctcaagcaatcctcccacctcagcctcctgagtagctggatctacaggaacacactgccatacccagctaacttttaatttttttgtagagatggagttttgttatgttgcccaggctggtctcaaactcctgggctcaagtgatcctcccacctcagcctcccaaagccctgggattagaggcatgaagcaccgcacccagccttggtgttgacacttcttggtgcctgatttcccctctgaacttcatgacaggcctttagggccagagggtcatctctaacagagcccaatttacagatgaggaaattgaggcccagaggcagaacagtgttaccttgtgggcccttgagtcactgcaaaaggagcctgtttggctggtcatctctgtcacagctctcttgtcacttattaacttgttggcttccttaagaggcagacagggaattccgaacagacactgggccacacggggcttaagcatgcaggtgccaccgttactcagatcccagttccagccctgctttcccacttaagagctctggaaccttgggccagttacttaaccactttgagcctcagtttctccctctataaatgggcgataataattcccacatcacagggtggttgtggagaaagtaaagtgccaaacttagtacctgctaaatagtaagcagttggtaaatattagctattattatttaagttatccctgttctttcctttcattcacatttattcaatgttttgtgccaagcacaagtgataaaaagtcccacctttctgggagacaacagcctaatctagaagccaaccaagtaaatagttataatatagggtgatagggactcgaacgggactacattctgcgtccaggatggcaaataggtgccatctctagtcaatgagtagcagctccctggagtgctgctttgaaaagcattctaaagctgtatccaggattgtgggaaagagtgctgtgatcaatgagtgacttctgccaccaatctaggaaggaatactaacagtacatgtaccatccttgccgtaaatgtcataggagcactgagaacagagcggctagtttagtcttgggatagaataagggatctgagccaggcatggtggtgcacacctgtagtcgcagctaggctgaggtgggaagattgcttgatcccaggagttggaggttgcagagagctatgatcacaccactgcactccaacctgggtgacagagcaagaccctgtctctaaaaaataaatttaaaaaaataagaggatctggtaaaaaccttatagaagggatagcatttgagtcttaatggatggacaggaaagtgctagaaagaaagaagaaacagcatatgaggtatattaggtgagaggttgggtaggaagaattacaaggatttttctgtggctgttgcccagggtagcagtagaaatcaggctggagagggacacagaggctgaagaggtaggcagtcaagggccttcagtcctcagctcatgagtgacctgtatcctgagcactggacatgggttacctgaatcccgagcacacatttcccacctcccgcagcatttccagcggccccatccagaagcctggcatctttatcagccatgtgaaacctggctccctgtctgctgaggtgggattggaggtgagtgacgctgggccggccccagtgggggcccactggaaattgggtcagactgtgatcccgcggtgacaggggcaggtgcctttccacatggccctctttcagtggacactgagggagtaggagccctacccaccctgcagagaaggcttcacagactggggatgtttgacccttctgcagccttccccagctctgatagttgttggtcacgagcttgggaatgtcgtaatggtaatcaaagagcggaccttcagtgtccacttgcatcagacattgttccatgccctttacacttcacaagaatgctgagggttaagtaccaccattatccccactttacatatgaggaaactgagacccagagaaagtatatgatttttccaaaatcgtgtacctcatatggtagggctgggattcaaacctaggtggtccaatcccaaagcaggaacccttaacctcttctttggacaagttacttcatgtctgtttgcttacctgtaagatggggataatacaggctttcttccaaggttgttgtgtgggttaaagtaattaatatgtatactagagctgtgccaagcacattgtaagtgatcagtgaatgttagctattctgttatgacgattcaacacagcctgtgctaaatgagaagctcaaaggttcccgcatctgcaaactgtgatttttaaagcaaatgtcatcaaatttagccaaagaaatgaacatgtaatggtataatatttgatagttgataagatagttatgaagtaggggagtgccagggtcagctttaagccctcccagagctccaccagagctttccaactgctccattcatcaggaggagctgaggtactgccacttgaagatccagcatctgacccagagcccctgggggagcctgtcctgcagatgagcgagtgtacagatagcgcaaacacactaatctttctccatttccccaccagataggggaccagattgtcgaagtcaatggcgtcgacttctctaacctggatcacaaggaggtgagatgtgggggtcttcacctgttggcccttgtcatctccacaccccacttctcatccccaccaccctggagcctggggccttctgtgctctctgcctggactgctgtggtctgtcaggcctcggcccactgtccttctgtccccacaggctgtaaatgtgctgaagagtagccgcagcctgaccatctccattgtagctgcagctgtaagtccagaatgagctggtgggagccccttgaccttcatccccagcccctctgacctttgatctctgccacacactcccagggtggctggtctccttccctgaagctctgacagagcagagcgagaggacttctgcccagcaagaagtttgggtcagggattgcgggagccgcagtgcctgatggtgctgagaagaccacctgcatctcggcccccaggggtgtgtcaggggatccccaggttccccgggggctgagcaaggggcctcttttctcccatgagggccgggagctgttcatgacagaccgggagcggctggcagaggcgcggcagcgtgagctgcagcggcaggagcttctcatgcagaagcggctggcgatggagtccaacaagatcctccaggagcagcaggagatggagcggcagtgagtgcagccagccctggatgccctgtcccgcctcccaccccaccacacgaccccacctagcttgcttcctgcccgctgtgtccccagccaacttcctcctcctccctggaggccagtcctcagaccagatgagtttggtggtaggtcagcgtatccatccttggcctcagaccacctggctccttcctccttgctgagcagagccccctgtcttccaacattccaagaatatggaaaataagcatcctactagcagtaggctctagctagctaggattagctaccagctaacatttgtcaagtacctccataaggctggtgttgtattagggccttgtttatgtttcttaattcccacaatagccctgggaggtagagagtattaaccccattttagaggtgtggagactgacactcagagaggtgaagccacttgtgtctaacgtcacacgtggccaagctgggatcacccccaggcaatctggcaagtccccacagggctgccctgcctatagtgatgaagctcacccttgtccaggaggattgaaatgatggcctaagagaataaatggggtgagcaattcataaatcaaaaactactgtcaagatccaaatccaaataccttttaggcatttaaaagtatttcatggctggacgcagtgactcacgcctgttatcccagcactttggaggtcgaggccggcagatcatctgaggtcaggagttcgagaccagcctggccagcatggtgaaactgtgtctctactaaaaatacaaaaaaaaattagctgggcatagtggcatgcgactgtaatcccagcttctcgggaggctgagacatgagaatcacttgaacctgggaggcagaggttgcagtgagctgatatcgcaccactgaactcctgggtgcaaagtgagactttgtctcaatcaatcaatcaattaatgtattttgaaaaggaaagaggaaaggctgtccccatctcccccaacacagagttagctgggagtattccacctggctaggagcccctgctttgctcctggggtcagtccaggccccgcctgtcatcagtcaccttacctaagtgtttggaggagggtgcatggagtgtggccttcacatggatctgcttccctcctcccacagcccagcatctctgctcagccatgccagacaaaaccacgcaagagcacagcgtccagactttgttagataacgtcccccaaaaccaaagctggtccaggcctcctaggaagggagcctggagaaaaatccaacttttctccaaatcaagaattcacagtaaggaagagttcatttctcttgcatagggccaaacatgccaatctgcatttgtgtttcagaaggagaaaagaaattgcccagaaggcagcagaggaaaatgagagataccggaaggagatggaacagtgagtacctcggctccacgcgtgtctgtgcatgaacatcagtgtgctcaggggagtgtggccaaccagaggctgcctccagaaccagtttacctggttctctcatcccctggtgggtcctcctttatttgtagtaaagcctgtcatattatagtaactgaaacatagtctcgtataattgccaaggtggggttcacactcaatttagaatacaagctcggggactttgcttgattcatcatgactagaaccatgaggcttctccccaggctggctggggctctccgatatgcaggagatgggcctatgggggttctgactccagtaacaggcatgggggtctcattttaggattgtagaggaggaagagaagtttaagaagcaatgggaagaagactggggctcaaaggaacagctactcttgcctaaaaccatcactgctgaggtacacccagtaccccttcgcaagccaaagtgtaagtttcatgagccgaggggagaggctaagggaactagtcagaaatgctggccctccctcccctcaccaccacctcctagatggatagcccttggtgctctgggctgtggttccttcatggaggggcagctgtgggtcagagaccatctgccccagcatcgaggtaggagggatctgtctgctccccttgttcacgggccagctccacatacccagctcccaggtcccccacaacactgacatgggcaggctgtcaggctgctgaagagggaataagggccatagtgaaagtggattagctcatgggattaccgtcctaatgttttgggtaccatgtcctcctcctacttggttctgaacaggggctggggtgaagccagcagcagaaaagaggggaaggacctcacatcagagaagggctctggtggtccaaggttgattcataactgtgggaggagcttactaagtgtctccagcccctatatccctgtatgtggaccaaggatggaggcaggaacaggacaaggaggcctctcccagacacccagctatgggctgtggctgtgctgtcctggggcctcagctcatactctccttaccatctcctctcttcatccgtcccacatcctcacctccattttcagtctaagcctctaccacctgctccctgaccaccttctcaaccccagcttccatatgcctctttaagaggcagcccacactgccagaggaaaacggggaccatgacaaccacaagtccaggattgctggttgggtccttactcctgccttccagctgttttgacttatagttgacacaggacaggtcctactccagctgattgtgctcagctgacctgggaacctccagcaaggggctattctggactcaagagcccaggttccccatctctgctgtggggcagtcatttggcatgttgtgatggggtctggacagctctgtccccaactttgggtcacatatgagacctatgggaatgtcaacctgccaacataggccacttgcaccaaggaagaggtgcagggcatctggatggttccatttcacccctccctggtgcagtcaaacagctcccaacacatttcttcctggcagcagggcagtgtcatctcctggtcctctcagaaggacaaagcacactctcatcagcctcccccgtgacattcagtatcagttgaggatatggccagagctaagatcccaatgaatgatcgctgttttagacagacagctaatttgctcttacgagtgaaataggagcttcaggagagaaatcgattttaattgcttctcatggaagtaatcctagtcaatttggtaccttccaagaaattgggcctcagcttcacagcaaacacaccctctcaggagcaatagaaaataaaaaccctttcactagctggttatttatttagtgccttttaaacaaatcaagctctttgaataaaaagaccaagaattttgcatttgctcaaggtaaatgtgatcttaggcagctccacaaagcacaggatggatgacccccgcctgcccgctgagctgggacagctgctgcctctatctgtctctgtatgcaccagcaatttaattctcatttggacctaaggcaggaaatgcagtgaggtccctgagccagctcacctcctgcctcactccctgttccccggggtctagcatggtcagggctgagttggtccagcaggcctgggccccagccagctcctatccaaccacccttcaactcagcacccagtttgcaataggttatacctgactcaggcttctatgcctgcaaggggtgggccctgcttttttttttttcttgagacagagttttgctcttgttgcccaggctggagtgccatggcgtgatctcagctccccgcagcctccgcctcctgggttcaagcaattctcctgcctcaaagcctcccgagtagctgggactacaggcatgcgccaccaagcccagctaattttgtatttttggtagagacagggtttcactatgttggtcaggcaggtcttgaactcccagcctcaggtgatctgcccgtctccacctcccaaagtgctgggattacaggcatgagccactgtgcctggccccgggccctgcattttttaaaataaaagagcatggggcatctcttacctagaaaatgaagtcactcatcctaattatgtgcctgggactgactgccccccaacctgccccagggggcccttaaaattgctcctgcccacctacccgtggctgatccagcagacccccaacattttgccaagccctgaaggccaggacaccaccacagaggctcctaacagaccttcccttagcttaggcaggagttatgtaggtggttatgcaggagttgccaggggcagccttgactaaggatctggtagctgaaggattcttgaaagatggagatgtttaaataggacatctccaaccttctctccctatccaaggccccatcaactatctcccccacctccaccacaagagaccccacgatttggaagttgaggttattttcctaccgaactattaaataatcatttgtgttccattgttttattaagtagtccttgttagtaagggagcatcagggttccactgttgggtaaatgtaatttgagccaagagccaaagaacttaacagcctttgcataggccaatgggcaagttctgttaagcttttaaaatattaaaaagcagctcacaagcaaacgggtatactaccttccagagtcctagggaggccggaggccagagtccaagctggaaactctggaatggagggtttgctcttctctccacattatgtcaaaattcaggtcttcctaactgcatgtaccccttttaccttttgggatgtccccacctcctcaggaccttcagcctccatctgctgcccacactgttcagacatcccctagggtcccagagctccaaggcaagggtgataagcacaaggctggagggtctgttctcccattgcagccccttgccctcaaacgtgtgcgattctcagatattcccatcctcctcactgcacttcccagccttcagcccaataccctacaaaatggttctcatgctaccctcagatctgagtttcctgttgttagccctttgtacagacagaaaaactaaagctgccaaggttgaaaacactgtatgcaccagctcagagcaaaaaacctttgctctgctctcctgaactctgtcccctgcccctgacctcacagctcccatggaggaagctgattacaggtccccaaactcctgagaaggttctccggcctcttgggctcccaagtcccttttggttcaccagcttcctcctcttttccctgggtagatgatcagggagtggaacctgagctcgagcccgcagatgacctggatggaggcacggaggagcagggagagcaggttcgcgtccccgctttgctccctggcctggctgctctgctttaccctgcccgcctcctcctgaccgcagtgcagacacccagcttcaggggcccagcatgtgtgggggccaatagagtctgtaaagctcctccaagccccagcttggcccaagcactgtacacaaagcctagacgacaggactcaatgcccaggctggtagatcagctgttgcacactggtaccactgaaccgctggctcgaatatttagatattgccaaattccccctctgctgctctggcccctcccccaggaccccctagaccactctcaaatccagcaactgaggaggtagtgtctggtccagcaggaggctttcagcatcctgttccagcactcaggctggtccggggtgggtaattcagccatcgctcttggccccagggagcttcagattgggggtattactctgtggagctgcggcctggggaaggaagggggctggtgcactggctatctggcctggatgaggatctgttttctgggggcacatctcctgcccgctcctgctggagctgtccttccaagagctgtcccagccactgccttctctttgaatgttgaaagcggaggacttgacctgcactatgagacaagccagttttgttctgtcgaacaacagttctaggcagacacccaggctcccttttgtctcgagccattttttgtatggagggaccagacatgggttagaaaaggccttgttctctttcaacaaggctttgtatgtgaagactgtgatttggaaaagctcatccttccacaaggggttttctctttgatgttctggccctgacttctgaaaccagtgttctggggagaggcatttgagagtgacccagggcctgggaaccggggcttttctcggtggtcttaggcctgttctgcaaccaaggcaaggccaggtaagccgagttggacagagggtgcagttttgctgtcggatctgccaatgctgtttatctcacaaagtcatgcattccttgggaacccagctcccatctgctgcccatggatgctctggctgagctgaactgtgtctcatcattttgttatctgtgtcttatagcttttggatggttttatcgttacgatggcaaattcccaaccatccggaaggtaggacagggttgggtgctgtgctggtgtgctgcttagtttgctgtgggttttggcttttcccaaacattccttgtcctatcaacactaaatggtgtgacttggcctgttcacttggaaggctaactccatctccatctttgtgtcaggctgacatcaaatggcagggtgctgttgcaaaggcaacattgggagagggatccatggtgtgagacctatccaagtaccatctggcagtgtgtgcatcagcactggccctgatgggaacaggaaaggggcaggaagcttctcctgtgtttctctgtcaagtagaactagagaccaaggccagaagcttggggtcagacagagcaaattatcggagtgggtgtacctagtgcatgttatgagaccagctgtgccagacgcagcctggctgacgaggtggggatttgctgcctcaggttgggacagagacccactccaagcacagccctctcctaggaatatctctgcatgtgcccaggataatgcatttttccaatttctaggacctttaattttctctctaaatcttattacaatatctccaggccttgtaaaactctcttgtccttatctttctaaatcacattgacatttgattgtgaaaacattgctgttaatatctataattggacattcgagatgacttggtatttggaggtcaggcagcagcataaatctgggtgagctaaattggatgttatcacttgaccagcgctttctggcaagatgtcagtcccccagaaaccagagcactggcagctgagtgaccttgatgatatataattctccctgcctgtaaaatgggaatattaactgtgccaggaaaatctacccccttcctccagcacattgctattaaaataacaaacagtaatatttttcctgacagaggttaatgcctttatctaggaacatctatcaggatttgtatagcattgaataaacttggaacagagttcctctgggaattccttcacacattgtcctggtctcagaaaggccttggtctcataagactatctgtttcccttgacaggctgactttggtggcttaggatgcctcattgggttagatttctaattcttctctatattctgcctctactagaactcagctagagaggacaaaacacacacacacacacacaagcacacacacacatgcacacataggattttacttgaaaaaaataataaaggagacagatatgtcaaatctttttcaggcactaataacatgtaaatgtaaaagaactagaatcttctccacataccacctcccatcagaaatcatgtccttgaaagtgctgttgataaagaaatagggttgcctttcccctattccttaatctaattattccagaaacagctgtcattttggttttcattttcacttcaaaaaaaaaaaaaaaaaaagaattgcttctgggtagaatcaatagcacaatcgccccatctgcctcacctctcagtctggaccaaaaggaatattgtaactgacaggccatggatgtaactgacagctgaaaggacaacagaaccatgcccctcatactggctctgaaaacccgtcatcattttttctagagccgtgagagactctttgtctcccagcagctggaacgccaagtctccaggaaatgcaagtgggtatccatggccatgatatctgtgcataaattccccttatttaaaaatataaagattcagctcatgtctataatgccagcactgtgggaggccaagacaggcgaatcacgtgaggttgggagttcaagaccagcctggccaacagggtgaaaccctgtctctattaaaaattcaaaaattagctgggtgtggtggcgcatgcctgtaatcccagctacttgggaggctgaggcaggagaatcacttgaaccctagaggcagaggctgcagtgagccgagattgcaccactgcactccagcctgggtgacagagcaggaccctgtctcaagaaaataataataataatataaagattcagtgtcttctatttccaccttggcagtaaatccctctggggctcaaggttcccaggcctttggccccacagaatttgtgccagggcatgaaggattcatgattctgactaagccctttcatcagaattgcttgagtcactcacacagaccagccctacttgcaggagcccaccgtctggcagggaaggcagattcacacacagctaagtaacctgtgtggggccagtctgagatcagtgggaaatgccatggggcaacttgccctctgcatctgtacagatatgactttcttgggcatgagacagaaactgtctactaccccacccagaagccctgactctataatcttagcactggtagttttttttttctttctttctttctttttttaagcaacagaatggccattattctcgtggtggtcctggaggaggccaggctcttactctagcaagctaccattaggcagtgtttctggctcctcttgggaaattgctgtctgaatctctactacgatggcagggattcatctatcaactgagtctagtgaacaaggtaggcaaggaaccaagcaggactctgaagcagtgagaaccattttctcgagaggctcatacactgctctggaggtgggtcccagaggtgaccatttccagagcacattagcagtacacagaggtgtgtgtctttaggagaggagctaggaagagtccctcagaggtccataatggcccaccaagtccctccagctgtggatgaatgttgtgtgtccattgctagggagctcaattccttattcctggccttggatactaagatgattcagaaaattcaagagcagatccccagaaggcttgttgggggcgggggtgagctcagtgaacagggggtttcctccttgccccagaaattgcccagggtgtgggctgtctctgctgcccctctcctgaatgctcgctctgtcctccacgctgtgtgtgataggggctgaccctccctcctgccgtgtttcccttgcccccagctcatcagggcaccatagaaaaactaggacctcgaagactcaggtccaaatcctggtactatcagttacaagatgtctgacattgaacaagggactttacctctctgagcctcagctccttcatcaaagaatgaggataaacctacataggagatcattggtataaatcccttggcacataggcagttcataaatgatcattctcttcctgccttagcccctggcttccctgcaaccccatgagagaagcagccaggaatgcaggctcggtgggtctaattcctacaggaaaaataaggagtctatttgcctttcatgtccctccccaaatcctctagaaccctgatggtgagtgtaaagcagttaagccattctctccatgccctgcctcccagtcctgcagtttccagctagccctcggtagaggggcacaattcagaggaaactgtgggtctagactctccagggtgaaatggtccagagcttaagcatgcgggtaaaatctgatgtccctgggtctgagcccccaggtctcagtgtgactcctatcacttctagcttaggacgggacctctacagatgaagcttgccagccctcccagattttctctggaatctcccaggatgcatctctgaatctgaacatgagatgatgcagaaggcatgtgccagatactggaaaatcagcacaatgccctttatttatatggcacttatttatttttttattttattttttatttatacagaacttttacaaagttctttcatggatgttatttcattggcactgggtggcaagcctgtgggggaggtatttttaccattttggagatgaggcaaatcaggcccagagaggttaaggaacttagccaaggccacacagctagttagtgaactaataagccagtctttctggctggtaaagagaatgtgtctcccactctgcagtaggtagcagtggccttcctgtcctaaacctgaggtctgatgtccagagtggtgagagagctgggggaggggctcaccatgggatggttgtgttatgtgaggactggatgccccgtgtgctgtgctgtcgacactatttctaacctttgtcatataaacaatgccacatctactcagaaaggaaaagataagaagaaagccaagtatggcagcctgcaggacttgagaaagaataagaaagaactggagtttgagcaaaagctttacaaagagaaagaggaaatgctggagaaggaaaagcagctaaagatcaaccggctggcccaggaggtatgtgtcctgcctgcaggccaggcaagcaaccctgggacacaatgtggtcgttctccatgtgctcccagaggaccagctggagttgttctcctcaaatggcatacaagggtggagtgagccctttcctgctccccagcctggagagagagatcatgccacctgcccagggccatctgaggttagctcctggcttaggaattgaaactaatctccttgcttgggggtgtgagtcttgctcctggctttggaattgaaactagcctccttggtcggggggtgtgagtctgaggttagcctttctgaccagatggtaggtctcaggttagcctttgatacacctggcttagagatttgagtctgtcctcttcactctaccccaccagagtctaaggctgggcctctccatctcaggtgtggctctgaggctacttagagtatggtctgcggctaacccttctgccttgcttgtagctttgaagttaggcttccactgacctgtctacgagtctgaatcactctggcctcccttcccaggttattgtaagaccagacccccccccacccaacttagcttggctctgagaatatgggtctgaggctaacccccttatgcaggtggtaggtgcgaggttatctcccaattaatcaggctatcagtctaaggctgctcccctcaacatagggtgtgtctaaggctagacctcatcaacctgggaatatatctgaggctgtccccatgaagacatgtccaaagcaaccttacaccatgaataattcctatgctaggtgagatgggtaaccttaactccccagctgtaggaggtatcattgtttctgcttctctttaacgcatccatgaatcagaatgccaatttgattagattataaaaccagagaagtcctggaaaatgtgaaatgcattaggtctgacattatgaacaaattagccacaaaattaaaaataagattgaacttatgcttctgagggcaaggcagaatggagcaggaaaaaaaagagtcaagttaaactcttggaatgtgctgggggaggctacaaaaactccattcttggaaaatcataagagcctcagggttcatggggatctgggatccagctaatccctcacaatacataagccccttttctgattttgcctacaagttctgcttagtgaatttgacaccccgccactctcttctcccttgctgctgcctccttgagggccagttggaacaagcccaggtctctttctagatagctgatgcacctcacagtactctggagtttctaaaggagtgctttcatgctatttctatccacatgttattcaaatgtgccctttgtcctctcctttctgcgccgatcctgggtcccaggtgtctgagacagagcgggaagaccttgaagaatcggaaaagattcaatattgggtggagaggctctgtcaaacgcgcctcgagcagatttcctctgctgataatgagatttcagaggtaacagagcccttctttccacatagaccctcctgctgtcttcagaatgacccactgtggggacagcgggaggtgagatgacaactagcaaacgtcactagcctcacagtgcccatccactgtccaggcccacccctaccaccccactcccctcttgaggaggagggatatgtctgtatttctgggtatactcccagagtgatctctaagtcccagctcatctgcgatagtctcagttaggcctgttgtcctggcatcatgactaagagtcccccttacactctcaagggcattccagtttagagaatgaactctgtgaacaccttaccacccacagatggcataacttggggctcttctgcatttgggcactccctaacagcagcctagtatggcctcagctgggcatccaggtggcagaggaatggcgccccatggttctgatgtaagggtggtgggtctccagtagcaagagaaacagattagaagagcatagtgctcgctgtattgtgaagtggagctctaagcagagtgacaattacagaacttccttgcaacacccccagatgaccacagggcccccgcctcccccgccttctgtgtctcccctggccccacccttgagacgcttcgcaggcggactgcacctgcacaccactgacctggacgacatccctttggacatgttctactatccccccaagactccctctgccttgcctgtgatgccccaccctccaccctccaacccaccccacaaggtcccggcgccccctgtccttcccttatctggccatgtgagcgcctcatcctctccatgggtgcagcgcactccaccccccattcccatccctcccccgccatccgttcccacccaagacctcactcccacccgcccactgccctcggcgctggaagaagcactgagcaaccatcccttccgcactggggacacaggcaatccagtggaggactgggaggcaaagaaccacagtgggaagcccactaactcccctgtccctgaacagagcttcccacccaccccaaaggtaatgtccctgttctgcatgctatgtttggaagtaggaagagtggggagaactgctgtttcccatgtcctccactgctcctgagagtggagacagaaagaaacatcatctcaccccattttccaggaatgccccctccgtgtgcatgtgtggacatcctgcatgtatatggtggttctcactgattttgaagtgctgtattgtatggtgaatggcctgggacctttggggtaagcaacggtgtgtccatttgatattcattgctctctcttagctcttctttgctagtgtaggctggtaactttcaatgtaggacttggaagtctatgttaatgaatagccagcactctgtcacatccagtcacttcctgatattatcccagaaagccttgaatatgggaaatggctcctgatttaacgggaaaaaaagggaggggaggaggaagcaggggcgtatttggctctgtaaatgaagtgataacactgtaccatcaaatgcagtattaggacattccagtgccattttctacagtactggccaactgcactggcttgaagtgtaatacccagaaatttctggccaatttccagctatagaaaaaatgaagatgagagagttcctagaaataatttactattaaaagaataggaatgtttttcttggatgttaggaagatgaacagtgtctttctgaaaaagacatatttcttattaattattcgtaatccaaagactattttgaaacatgcaatctctgaggaagacagggagcttggatcccatgaagggaaaaacaaaatattattccactcccgtagccatgaggccaggcttgagtcagtgcaaaggcatctcatgtcagctgagggtagcaggggttgctgagggagggcatagtcaatagcacctgaccagtggtttttattaaccttccctgcatggatttgagttcctgcgtggagagcaggacaagcatctcagagcactcaggagagcttacaaggaaggtgggagtgaagaaagtgccagatgccagcaaggctgaagcttccagcgggagactctcagagggaccctttctggacctctggttggggtcgtgagccctgcagctttgacaggaagaaatctgcctgttctcgggacatctttccccagagcccagccaggagctggtttctagagctgagttagccttgcacaacatttttctctgtaagtggcctccatcaagatgagcagtagcttttcctcaacaaatttgattccactttgattgagtacctgcctggggcctctctctgtgaaggggagaggggagcagtgctgaagaggatgcagaggagattgacatgcctgtgctttccaggactttgccatctgaaggatgagctgctgcccaggcaggcttctgctataaaatatgcataagccatgcgctatgggagggattcattctaactgaagattgaagacacctctgtagagtagagggcttacgagctgagccttgagggatggcaacacatggaggatgggagcaagggcattgcaagtgcagggaacataaagatggcaagttggagtagcagggagttggcctgcctgggtttgaatcctggctgtgccacttactagctaggtaactgaacacatgctacttcatctatctgagcctgggcatccctgtccaaaaataggaatgctaatggcagctacctattcgaatcaaataagttaatacatggacacattctaagttaagaatggcaagagtggcaggcacatcgtggatgttcagtagacattaactatggttattgatagtccagagtaaaagaaaatttgggtgaagctgcaaacctgggatcttagcaatctcagtgtcagcaaaactttgagtcaggatcaagattcaagacagagtccagacttctcagattcttcaaggcatcaccaagttcccactgttagttcctgatcagagcccagggaaggttaaaagccccccaacacaaggagcctctgaagaactggactataaatgtcacaagcgacatcaaagccagggttgtcccagcctcaaaagccagggggctgttgcttggttggcttcatttggcccacctctcacccttgtaccacccaccctcctcctccctgcatgcccctggcaagctgtcatcagtgctgcagcaggaagtgtgagtgaccacacgcccacacagtcagcccagccaccagcaggccccaagtcatggaaacaaacacctgtgttggcttgacacttgtgcctgccacaccagcaccggccataggggggttttcagggccagatctatccacaggcactgtcccatgttctcaccatactatttagtcattcatttattcattcaacacatgcttattgagcacctactatatgccaaggcactatgcagggcactgggaatccctgctggatagacagctgcagtctcccttctcaagacagtctggtggggaacaaagaaaagtaggcacccgccacaccatcagcgtggagacggaggaagcacaggtgctataataaggaagagggctggagtagaggaaaagctcaagggaggcttccagaaggcagtgacgcccaacttaagacctaggaggcaaagcggaagaaggaagagcacgtgcaaaggcctcaaggcaagaaagcctggcattttttgaggctaaagctaggcagtgtgaggagcgtggcgaggagtgagagataaggctgaaaaggaagctagagaagctcatgaaaggccttgggagtcaagccaatgagctcagtagtcaatgcggacactggggagggtttgggggcagggctgtgaactgatttgatacatcttgcccaaaaagatcaagtgggttagagccaagcagacaggttgcgtcagctcccctggatgcctctcttactggtcttcaccccatggcccctagagactatgtgcctaccaactaggaacagcagatgcttcacactgtggtccagggactggaactgtggccaaagagacagcccttaaagacaagggacactggatagcacccatggccactgagagaagggtctgaaagtgtgtctcctgggtgacactggctgctggcacccccaagccaggctctagctcaagcgtccaatttcttagagccgctcagtagtttctgtgatctcagcccacactgtggccaatgggcttctcatctctctgctgaggatcacaaccccaggacctcgagcagttgaatgaccaggctggcttcacatatgcatgggtgtagatttgcatgtgtgtagaagtagcatggctggccttggccaccccaccatgacctcacctgggtccctgttaaactaactccttagacattttgcccaagcccacagcctccacgaggccctggcgtgtccaccatctccaaacctgtcatggtccaccaggagcccaatttcatctacaggccagctgtgaaatctgaagttctggtaagccccttgggtcccctccaggttgtctctagaggagcagaccagggctacctcccctgggctctgctgtctctggagggcaagtgaggtgggacaaaaatgcagatcacagatgccatctcttaaccttcttattggccaagagatgaggccacatgggtgtaagtggatgttgacatgaccagtggaatttctgttccccacttgactgaacagcctggagctctgtctaaaagtaataatgatcgctacgacctgtgaagagcctactatataccaggctctgcatggcctcaattaatcttcacagcaggactgtgagatggttcctgtggtcatcctgtttaacactcgaggaaagggaggtttagagaggttaccagaggggcaccaggttccccaggcagagcctggacttaatacttaccctcatgcctttcatactccaaagcccacgctgttaccctcgttgccatcttgccaaaacctacagagattccaggtgccctggcccctgccctaaggcctgttgtggcctggcatgaaacaccctcagatgggaaactggccagagcaggttcactccctttctccaacttacttctcacctctcactgtgactggaagtctgaggtgtggtcctggggaagtgagaaatgtccgccagtctcagttactgacggctaagggagctgggattcgtgtgcacctcccagaggtgccgaccactggctggcctcccatgcacagtgagaagacagtcatgtcagaattttaacttccctttcaaggaaactctatccaaatgtcagggcaggacagctgagatatttattttgggccttaactgtcccgtcagctccccagagagcaaagtttttgctcctgggacagcctgcggatgcatccatgtcatgttctctggctaatatcaaggggtggtgtctgcttgacaacaactgggtagcatctttgggcattgagcaggcctttaacgtaagccagactgctcggggagggacattggcacggcagccccagactggcaggcccgaagcctggcaccagggggcagccaagacctattggtaacatccaatgtggaacttttttttttttcctctggcagccacaggagatgttgaagaggatggtggtttatcagacagcattcagacaagtaaactgatacccattgtgtgtctggaggtctccccaccacccccgtccctcccactctgtgccacttctttctctctgggagtacctggtcaggtccatggtggccccatctgccaccaagcctcaggccagagctgtgtcctccattgcctgcgcaggggtgggggaggatatcatatcagatggggacccagggcttattagaccccatgcatgagctgagaaacagcagtatccctgggaactcacctcttctgctgctgtttgtccagagaggagcagggcagaaggaaccctcagaggcccacagaggccaagatgggccctcgctccagccctaggagagagggaaggggctgcttaggtgtctccctgtaccttcccctcttcttctccgggcagatcacagcttcaccaaccctgcctgacactgaaccagcgtcagggaggaggtggtggctgaggtcctccctgtgcctagggcactcctgtgcacagggacgtggaagtccttgctggtcctctgagtggcatcaggcaggcagccacctacctccctgcctcctggggtacctccatggagccaaccattggattggctgtttcatcaccaatgaaagaaaccccaggacagagagagatcttgcagccagcccaggcttctccagcctcctcactgataacccaccaacagaccacaagctgatttgactacaggcagggagcagaggggcagtatcaactgtgcagtgagacataggtgttggcaaaagaggctgctcccagcccaaggatttgtcaatttaatctttccagcaaccctgggtggtaagtgctattatcatacccattttatctatgagaaactgaggcagagagatttgaaaatgtgcccacgatggcacagctaatgtcaatccctggatttgaatccaggaggtcggtctctgcagcctgtgctcttaaacactgcaccatcctgccttaaagcactgtcgtcacacaagaggccttccaggacctggcggtttagggcacaaacagccctcacagggagagaagcagggaagtgcagagtctaggctccagaggtggagtcaaacccttgctcccctgcccactgactgggtgaccttgggcgtgaatatcacctctctgggcctcagtttcttcctctgttgaatgaggggttgggctgcctctttaagggccaaccagctgcagtggcttatgagtctctgatctaaattcacacaaaacacaagtacctgccatctggcccatccctgagagctggttctcaggatcatggagcaaggaggccagagccagcactgcccagccttccaggagaaacagccggagtaggcagggccctcaaagtcaaagagcatcgactccacatcctgcccaatgatccttcttgcctgtgcattcactgcaccccctcctctgtctgctgcccacacactgacctggatgtagctcccaagctgagccgagctcatggcctcttggggttgagcctgggtgattgaggcaagtgaggagggatgccaggcagatgcttggggatctgtctgctgatatttggtgctcatcttgtgcccggaacctagttggtgcattctgaggataggggaatctgtagcctccccaccacacagaccatgggccccatgggtcacttggtgtggtctagggacctgcatgctgtgggtggctccactcagtctggcgaggcctgccacggggctctcctctgctcccaccttccatggggaccagccgtggctctgctgcccttgtgcttagcatcctggcccctcagcctggggatgcccctggctcccactcactgtgtgtgactcccgaggaaggccacatagttcagggactacctcactgtgtgttcaggtgccacctcactgtcttgtttctacaccctggagccattgtccccacagcatcccccatcaagccaggtcccctgagctgtgtgtcctcccttctcccctagctaaagggccaacctgcgctccgcagaatctggggaggcctcttaccttctaggtaagcattacatgaggacactgccaggctccaggccaacactgcttctcaacaccacctgctttcttcttgttgtccgtgggctccctccaccatcttgtggctgatttccaggaacagccttctcttgagctggatgggcccctcctgtggggtcagagcactgggtggagacaccttgggcttcatcccccagggcctgggcctgctccagtggagacttggaagagatgaggtggggcctaaaggacttgaggctggggctgagactttcagcaaggcagatgccgcctctccagaccatctagacgtcactggtgcccctgcagcccctgacgcttgtgccgctgaagcagggcagggtcaagatcctctaaagtcttcctcagcctcctgcttgtccctgaacaggggtgccctggctgctagggctgccggcctcctgtctgcatcccgtaccctggccgtgccttctcccgccctacccctcacttctgacccttggattccgaccattcatccccctactcctcggctctgaccctcaagaccctctgctgtgttgccctaaagccctccctttgcttccaggatttccggaaatatgaggaaggctttgacccctactctatggtaagagatgacgcttctctcctggacaagtaaccccaggaacagggcagtgtgggggttagagggtttgatagtggtgcattctgggccttggggtcctgggatgaggtggggcacagaggagccccagtgatgccccagctgctcttcccacgaagattccttccaaagggcactccagtgtgaactgtatggtgcacatgcacgtgtgtgtgtccactgtccccatggggcctgggacccccagttgaagaccagtaggggtggggctgggcacggtccccttgcccatgtgctctgtggggcccagtgtcccataggtgcctggattccccttccagccctaccccaagcgccatccttcaggccagctcaaagcttcccactgtctttttctctctagttcaccccagagcagatcatggggaaggatgtccggctcctacgcatcaagaaggtacctgggcatgtggaggccggtgggccgccatccctctctgtgcctgcccctcctcccttggtctcctgcctctactctcaaggtcactcctgggtggtctctcaggtccccactgtcctcccctctcccacggagacgcccctctgttgtagggccgcatacccaggccccacttggcaccaaggctgcgttttctccagatggcactgccggtgaccccatggcttcttccactcttactgtggctctgtcaagagactccttggtgcccaggtgtccacaggctgctgtctgcttggctccctaaggcctgttttcctctaaccaggagggatccttagacctggccctggaaggcggtgtggactcccccattgggaaggtggtcgtttctgctgtgtatgagcggggagctgctgagcggcatggtgagtggagactagccacacccaggtttggggatgatacagtggttagacggggccctcccggaaagcaaacaggtgaccacttggagtgggctgacggttgctggagaatgccctcccactcgggtccatccatccgactgcctgtccaatccctgggggcatgggatggcgccgggacctgtgagtacacagccaagccagatgccaccgtgccctcagggggtccccagccagggtgggagacggactcagaattgccagatactgaaagtcacagcatggtctgctaagggctgacggggaactcagagtggagggaagccatgggaaccctgaggggcctctaacccaccaagaggagatgggaggccaggagtgctcctcagaggggcgaaccctgagctgagcatagtcaatgtcagcctcgcgaaggggagaaagggctttcgaggaagaggagcagcacgtggaaacccccgaaaccttgcctgtcctttcagtgatggtaggagccgaacgtttggccagaactagccatgtgccagaagctattcatctggtttgcagccctgtgtctgagcgtttcatggatattaactcacttaatcctcacaacaaccctgaaagcaggttctgttattattcttattttacagctgaggaaactgaggctcagagaggttaaagtatttgttcaaggacccacagccaggagaaggtggaggcaggacttgaatccaggcagtttggtcattttgctttactgtccggcacaagtgccacctctgtctgaggtcttctgcagccttgtcccttcccagagggccagaggcccccataccctgtgtctctcttctcattttgccttgttctttatccccttatctacaaaaacaccctctgctgatgagtgaaacctttgcagaccccaggcacagtctcttgcttcaaaccatcaccccgttcacctgtcggacagtgtctgaaaccccaccattggtgatttcttcttccatgtcaaggaagctcctgagtcctttgtggccctttgtggagatgtaggtgcccttggaggaggagccccaggcctcttaccttcccaggccagagtgggggaccctatgagcagatcccttccaggcagtcctaggctggagccaggctgtaccataagctggaggtggtagaatggaggtggtaagaggtaaggggcaggaaacagggatgggaaaggcccttgggggctggcaaccagacctctgtgggttactaaggtgaggtatgggcattgggagccctggatacaagtccagctctgcacccatgaccttggtgacttcacactttaagctttcatttcagccatagaagggggacctgcccagctgggcagctgccccagcccatcagtgcccacccaggccccaccttcttcttctgtcttcatttctctgtcacctggtgacacttctgtgatacctgcctgctgtgtctagcagagagaggggaccaggatggatgggtgactctcctgggtccttcccacttacaaagccccaacccaaccactgccttattgcccctgagactctgtcatggatttgtacaaaatgaaaaggtgaaagtcacccatccccagttgcactgcacagaggcaactgctttctgtggtttggtcctgcactgctttgaacataactagtcactgatgaagcttccctaggagcacgtgtgttttaagccaccatggccaccattgtcagtatcatacaccacattcctatgggccttcatgccacaaggctcctgggtgatctatttcaatgtaacctggagagtgttctgtggacatcaggactgggagaaattcctccaaaaagggctctgatgtcaaagaggtgtggaaaaccacgcacaatctgtctacctcttggaaagctgcagtgcatgttagcacattcaagactgacaaatcctgcagtgagtagctctgttcagcttataactcagctttccccaaaattattagatcccaggcaactctttcaggagcaccttattacagcttcatttattcaacaaatatctactgaggtcctattttgtgtcaggcattatgttaagtgctggggatattatggtgaaccaaaaagacaatccctgcccacgtggagctgacagtgtggtgggagagaccatcagtaaacaaacagattaatgattacgaattgtggaaaatgccaaataagaaaagattgggatgctgtgagagaataggcaagatctacattcgactgagcgggggtcagagaagccccttctgacatttcctctcagacctagaagaagagaaagcaaggtagggaacagcgacatgaagcctctgaggcaggagagagcatggcagctcagggacctgaacagaccagcatgactggcaacagggagggccggctggcagaattgactcaggcctcatactgtgctggaaatgcaggtctgtgtccctctgtaataacagaggctcatcatcaatgatatccaggttctcctgagctggggtgagggggtgtggaagggacagggactaaatgccctttgactagccactaaccaggtggctagtttcccctcatgctttctgagtccacatgagctttcagaacccaggctcaggtctcttctgctgtctggcaatgacccccctttgccaagccctgggccaggcacgtgtcacatacagcccagttggccaatcagcctcatctgcctgcaggtggcattgtgaaaggggacgagatcatggcaatcaacggcaagattgtgacagactacaccctggctgaggctgaggctgccctgcagaaggcctggaatcagggcggggtaagaataaggcccctccctcctttcctccctcacctgcctgcctcaaaccctggcctctgcagccaggtctcacaataggatgcctcattccagggtgggcatctggagtccaggcaacactttggtgacaccataccccatccagcctgtggtttaaatctgacaagatgggattcagaaaaatagatgtcaattcctgaccttggatccaaaaagccagtggcttaaacagactcttgaagccagggcatggcaggtcacccaagaaaaagacttaaggtcttttctaagtgcacactgaacaagaatcaagagaattctgggggctaccagaaagcattaacaaaagcagagaacccaggatgaaggaggggctggtgggaatctgctctgcactcattagacacccacctggatcacggccactgcataagattgcccaggctgtgcactacataatctgagggggtgcccttttacagactgtagtgtgaatggtgcctgctcatggtgtgcaatgcacaacctgtgcaactgtatgcaggcagccgagcctgaggtattaagttcagtgccgaggaagctgaccagatgggcctgggacccacatgaagggatgtggagaagagaagactcacgggtaacatgatgactatctttgattatctgaaggtctgcattagggcagagggagcagaagtgttctctctgattcttgagggaagacctggattggatggagggaagtcctggggaaagagagagagatttcagctcaatattaggaaccagctcatggtagaggggcccagtgatatttccaatacttttcaggggatacacctaagccaaggagggagaatgaggggcaagtcaaggatggcagtaaaagggattcaggtgttaggatcaaaaagctgggccagaagtcccccacttgattgtgcatcagaaagtgcgtcggaattacccagagaggttgttaaaagtactatttcccaggactcacccttgacttctgggggccaagtctacgagcctacacttttaagaagttcccccaagtgattctgaggttgctgcctgtccccggtccatagattgacatttgggagctgagtgtcattcaagggccttgcagccctgtcctctatggtcccgaagcctcagagactagaaacgtcctcagaccatggaagtcacagtgggccccaggtgggcccgtgggaagaaggtgcagcctggctgatcctaggccatggggcccagaaatggggatggagtgccggggcagatgcaccatccaactgagtgtgccccagagtcacacggcttctccccacaggactggatcgaccttgtggttgccgtctgccccccaaaggagtatgacgatgagctgtaagtgtgtgcaagcacctagcctgagacctcttcttccttctccagaatctcagccacctttctccagcccatccccagccttctcccagcctgaaggaatggcccaagcacgcagcttctcatagccagagctcctagaaaactcctggactagagccaagtcattgtccttaagcagtgtctgagctgcctcccgggcagtcttgtccaaattcttctctactatggggaattgaggcatgaggtcttagaccgggtaagcagagacgttgggaaaaagactcaggattgttaattccccactcagaactttatcccctgccccattattagatggttagaaggtgtctgtgtccatccttcactctctggaaggtcttcctgatgtctaactgcattcactcatactgtgcctctaggcagccgggaccacaggcttttgtccccacgctgtgggatctccagggagctttggcctagactgtctctctctgtgattccctgtgtgtctgtctcttgtgtcctgtgggtctctctgtcttctctcctcctccctctctgtctttctttcaccctttccttccttcctgtcaacatcttatctgcccccctccttccccttcatcccggtcccctttctctctcactgtctcctattctttcttcctgtttctcttcatccctgtctctgagtccctgttcctctgtccttgtcattctgcatccattcttacctctttgtctgcctttctctgtttctctgcctctgtgtggtgtctcacctccatcctcacctcatcccatcacctccccagccctcacccacccaccactcacccactcactgaccatgccctgcctccctgtcgtggctgggcctgcttgctccccgtgcccagcagaatctgagctctacacatgtcttggagaaaccagggtctcgcagctcctaattctggaacccaggggctaggcagaacccgaggcaggagcccagtgaaaggagaagccccatggagctctgcctgggagtaaccaagcctgttttgtgtttcttgctctgctctgtatatatagagcttctcttccctcctccgtagctgaaagcccccaaccggtccgaaagctccttgaagaccgtgctgccgtgcacagacacgggttcctcctgcagctggagcccacggtgaataggcaggcgggccacagggccctgtgtgtccctgctgcttgcagtggccatctgctgcccacgctgtcagcaggttctttggactggcgtctggagggtacacaaggcgccatccctgaagtgctgcctggggcctgctgttggccacagtggaattcctcagactcaaagccctcccctcagggaagtggtgcaaagcccagtctgtagtacttgcttgggacccaggtgtcctgactcatcacggccctgggacctgctttgggtcccaagcagcacccaaatgagcaggatgaagccctgggcaacattctctgagggacacagacaatgcctcgaaccaggcatgtggggctgaaggagcccacaggaagcctggctggaattgcccccaagagatgtcctcaacagaatgtgaaaattccccttcctgtgaatgccaacctcctgggagctcttgctccaccatggcccccacacttggccagaaccagggctattaagaggttttgaaggctggtccaaagaaccagggttggtggattagagttgctcatgtcctgttgtgccctgtcatggcctgagccgtgcttagaccaaacggtcttctctgccttctcccttccttggctctgggctctatctgtgggatataccgtggaacccagctgtaggatgtgtttctcaccctgtgatagggatgtgcccgtggacagagctggcaggtggctgtgaaactggtgtttggtgtggcctgaagccacagatggcagcatctggggcagacccaagcctggacttgacttttctgttacaactttcccaaaacattagggcctcatgctcccaagacacttggccgggtagaccatggcttctggggtggcctgcatggccctatgttttctgttacttgccttttgcaaaggactcttgcccctgttcctcccagtctcctccttcccccatcccaggtgtccctcccgctttctccctggcctcctgtgtgctgtggctgtggctggtgtgtagccacttggggcctgcacccagaggggtctccaaagggtggcagggccttgtgctgccagagtgggtactcccccttgtggggccttgctctggctgggctgagtgtgcacccacaaagcctgtatccacctgggctgtttccacttccctgcaggaccttcttctgaagtccaaaaggggaaaccaaattcaccgttaggaaacagtgagctccggccccacctcgtgaacacaaagcctcggatcagccttgagagaggccacactacacacaccagatggcatccttgggacctgaatctatcacccaggaatctcaaactccctttggccctgaaccagggccagataaggaacagctcgggccactcttctgaaggccaacgtggaggaaagggagcagccagccatttgggagaagatctcaaggatccagactctcattcctttcctctggcccagtgaatttggtctctcccagctctgggggactccttccttgaaccctaataagaccccactggagtctctctctctccatccctctcctctgccctctgctctaattgctgccaggattgtcactccaaaccttactctgagctcattaataaaatagatttattttccagcttataggagtgagtgtggatttgggcagcagattcaaggctgcaaatcaaaaaaccataaggtttgtggcccctattcaagggtgatagacagatcccagtgctgtgatctgggtctgacatgaagggtgtgatcaaatggccagggctggcttggagcagaggttgagaaagcaggagatgggctgggctgggcttcaatgtcttctcagcagagatggtaggagatgaagtctgtgtggcagggattttgctcaattccagaaagcagagctgaaggcaggagccccgaagggtcacctcatgatatggggtgcccagcttctttcaagaacgacacagccaccaatgcttctcctgaggtcaccacgacagcatgtgagggaggaagatggcagggtccactccctccgtggaagcacatcccacagaagctcatgggaaatgcaagggcttgaggcaggaaggcataactccgggggcccagcagggggaatgtcacagttcttctggtgacagggaccagggctgctagctctgaggaagagggtggggctgtatcagcacgactcgcctgaccccgtctctgtttccccattccaaattcctgtggtacaatctgatcaggccaaatcacctgagcgggcgacccttgggttagggaccagtcacaggtccaatcagctgtggccgggggtggggtcacatcccacccaacatggctgctgaggcagcaaggaccgtgggcagcaagtcatgatcaccacccctaccagaggatgactatgacatctctcctcccccaccaaaccctgggtatggaaaggaagtggactggggtcccaagggaaggcagtcttagggagagtgcctctgtgtgaggggaccacaggaaaaccccttcctgaggtgggacactgcagagagcccctcggggacacacccactgcccatcccccaggttcagccctatgttcagctgaatcccccacttcatcccacagtggatgcagactaagctggatcccgagaacttggtaataaaacagacaggctctcaacacctcccagaatcatggcaggggagaggggctcagaccaaaatgcagcgactaccatgtgggactgaaagaaatcaatgggtggggacagagagagggagcagagaaactgccaaactttctgatgtcctcgatgaagacaaagccacagtgaccttcaaattactggcgctcagagacagcagccacacagacgagctccctgtgttttcctgtcaggcacctaacctggttctggagaaaattccaaaacccaggtaagaggagggagctcctatttgctgtcacccatgtgcctggcctaggctagacccttgtctgctgtttcaaactgcaacacctgggcttcatgcctggccatcccaggagcctggtgatagcagcccacactcagatggcattgactaagtgccaggtattgttctaagcactcagctcatacattccatcaacaactccatgaggcaggtagtattattatcaacaacctcattttattaagagacagttgatataaaaagaggtgaagtcacttgccccaggtcacacggtgaggaagtagtagagctgcgactccagccaggcactctggtcccagagactgtgcacacccgtttccaccatatgctctagagggcaaggcagctcccatcggaatgtctcaaattccaggccgttggcagaacacttctccccaccactgaagaacagtgactgttctgctgttgggtagtggtcacttcctcttgtgccctacaaaaacttctgctgaggccttcagctagtcagtgcctggaatctcaccattggaaattcacctgtagcaggagtgaatgagtttatcagcccttatccggacttggtgagtgagacggtaaagaggctttgagctcctgattagaggagaagggcagggaggatgagcactgggccgggcaggaag

SEQ ID NO: 2 - DNA, 25 bp, Artificial Sequence
  Description of Artificial Sequence Synthetic oligonucleotide
agctgatcatattctacctggtgct

SEQ ID NO: 3 - DNA, 25 bp, Artificial Sequence
  Description of Artificial Sequence Synthetic oligonucleotide
atattccacctggtgcttcagtggg

SEQ ID NO: 4 - DNA, 25 bp, Artificial Sequence
  Description of Artificial Sequence Synthetic oligonucleotide
agctgatcatattctacctggtgct

SEQ ID NO: 5 - DNA, 18 bp, Artificial Sequence
  Description of Artificial Sequence Synthetic oligonucleotide
acggccacgtccatggtc

SEQ ID NO: 6 - DNA, 18 bp, Artificial Sequence
  Description of Artificial Sequence Synthetic oligonucleotide
cgagcacggccacgtcca

SEQ ID NO: 7 - DNA, 18 bp, Artificial Sequence
  Description of Artificial Sequence Synthetic oligonucleotide
tcccacgagcacggccac

SEQ ID NO: 8 - DNA, 18 bp, Artificial Sequence
  Description of Artificial Sequence Synthetic oligonucleotide
aggtctcccacgagcacg

SEQ ID NO: 9 - DNA, 18 bp, Artificial Sequence
  Description of Artificial Sequence Synthetic oligonucleotide
gcttcaggtctcccacga

SEQ ID NO: 10 - DNA, 18 bp, Artificial Sequence
  Description of Artificial Sequence Synthetic oligonucleotide
gaccagcttcaggtctcc

SEQ ID NO: 11 - DNA, 18 bp, Artificial Sequence
  Description of Artificial Sequence Synthetic oligonucleotide
ttgatgaccagcttcagg

SEQ ID NO: 12 - DNA, 18 bp, Artificial Sequence
  Description of Artificial Sequence Synthetic oligonucleotide
gttcattgatgaccagct

SEQ ID NO: 13 - DNA, 18 bp, Artificial Sequence
  Description of Artificial Sequence Synthetic oligonucleotide
gctgggttcattgatgac

SEQ ID NO: 14 - DNA, 18 bp, Artificial Sequence
  Description of Artificial Sequence Synthetic oligonucleotide
agacggctgggttcattg

SEQ ID NO: 15 - DNA, 18 bp, Artificial Sequence
  Description of Artificial Sequence Synthetic oligonucleotide
gaggcagacggctgggtt

SEQ ID NO: 16 - DNA, 18 bp, Artificial Sequence
  Description of Artificial Sequence Synthetic oligonucleotide
aaacagaggcagacggct

SEQ ID NO: 17 - DNA, 18 bp, Artificial Sequence
  Description of Artificial Sequence Synthetic oligonucleotide
gcatcaaacagaggcaga

SEQ ID NO: 18 - DNA, 18 bp, Artificial Sequence
  Description of Artificial Sequence Synthetic oligonucleotide
gaatggcatcaaacagag

SEQ ID NO: 19 - DNA, 18 bp, Artificial Sequence
  Description of Artificial Sequence Synthetic oligonucleotide
cggccgaatggcatcaaa

SEQ ID NO: 20 - DNA, 18 bp, Artificial Sequence
  Description of Artificial Sequence Synthetic oligonucleotide
atcagcggccgaatggca

SEQ ID NO: 21 - DNA, 18 bp, Artificial Sequence
  Description of Artificial Sequence Synthetic oligonucleotide
gtgggatcagcggccgaa

SEQ ID NO: 22 - DNA, 18 bp, Artificial Sequence
  Description of Artificial Sequence Synthetic oligonucleotide
cttcagtgggatcagcgg

SEQ ID NO: 23 - DNA, 18 bp, Artificial Sequence
  Description of Artificial Sequence Synthetic oligonucleotide
tgcttcagtgggatcagc

SEQ ID NO: 24 - DNA, 18 bp, Artificial Sequence
  Description of Artificial Sequence Synthetic oligonucleotide
tggtgcttcagtgggatc

SEQ ID NO: 25 - DNA, 18 bp, Artificial Sequence
  Description of Artificial Sequence Synthetic oligonucleotide
acctggtgcttcagtggg

SEQ ID NO: 26 - DNA, 25 bp, Artificial Sequence
  Description of Artificial Sequence Synthetic oligonucleotide
atattctacctggtgcttcagtggg

SEQ ID NO: 27 - DNA, 18 bp, Artificial Sequence
  Description of Artificial Sequence Synthetic oligonucleotide
ctacctggtgcttcagtg

SEQ ID NO: 28 - DNA, 18 bp, Artificial Sequence
  Description of Artificial Sequence Synthetic oligonucleotide
ttctacctggtgcttcag

SEQ ID NO: 29 - DNA, 18 bp, Artificial Sequence
  Description of Artificial Sequence Synthetic oligonucleotide
atattctacctggtgctt

SEQ ID NO: 30 - DNA, 25 bp, Artificial Sequence
  Description of Artificial Sequence Synthetic oligonucleotide
agctgatcatattctacctggtgct

SEQ ID NO: 31 - DNA, 18 bp, Artificial Sequence
  Description of Artificial Sequence Synthetic oligonucleotide
atcatattctacctggtg

SEQ ID NO: 32 - DNA, 18 bp, Artificial Sequence
  Description of Artificial Sequence Synthetic oligonucleotide
tgatcatattctacctgg

SEQ ID NO: 33 - DNA, 18 bp, Artificial Sequence
  Description of Artificial Sequence Synthetic oligonucleotide
agctgatcatattctacc

SEQ ID NO: 34 - DNA, 18 bp, Artificial Sequence
  Description of Artificial Sequence Synthetic oligonucleotide
tcagctgatcatattcta

SEQ ID NO: 35 - DNA, 18 bp, Artificial Sequence
  Description of Artificial Sequence Synthetic oligonucleotide
gggtcagctgatcatatt

SEQ ID NO: 36 - DNA, 18 bp, Artificial Sequence
  Description of Artificial Sequence Synthetic oligonucleotide
gggggtcagctgatcata

SEQ ID NO: 37 - DNA, 18 bp, Artificial Sequence
  Description of Artificial Sequence Synthetic oligonucleotide
cgccggggggtcagctga

SEQ ID NO: 38 - DNA, 18 bp, Artificial Sequence
  Description of Artificial Sequence Synthetic oligonucleotide
tggagcgccggggggtca

SEQ ID NO: 39 - DNA, 18 bp, Artificial Sequence
  Description of Artificial Sequence Synthetic oligonucleotide
gcacctggagcgccgggg

SEQ ID NO: 40 - DNA, 18 bp, Artificial Sequence
  Description of Artificial Sequence Synthetic oligonucleotide
cctctgcacctggagcgc

SEQ ID NO: 41 - DNA, 18 bp, Artificial Sequence
  Description of Artificial Sequence Synthetic oligonucleotide
ggcttcctctgcacctgg

SEQ ID NO: 42 - DNA, 18 bp, Artificial Sequence
  Description of Artificial Sequence Synthetic oligonucleotide
ctggtggcttcctctgca

SEQ ID NO: 43 - DNA, 18 bp, Artificial Sequence
  Description of Artificial Sequence Synthetic oligonucleotide
ccagcctggtggcttcct

SEQ ID NO: 44 - DNA, 18 bp, Artificial Sequence
  Description of Artificial Sequence Synthetic oligonucleotide
tgcctccagcctggtggc

SEQ ID NO: 45 - DNA, 18 bp, Artificial Sequence
  Description of Artificial Sequence Synthetic oligonucleotide
ccccctgcctccagcctg

SEQ ID NO: 46 - DNA, 18 bp, Artificial Sequence
  Description of Artificial Sequence Synthetic oligonucleotide
ctccaccccctgcctcca

SEQ ID NO: 47 - DNA, 18 bp, Artificial Sequence
  Description of Artificial Sequence Synthetic oligonucleotide
gatctctccaccccctgc

SEQ ID NO: 48 - DNA, 18 bp, Artificial Sequence
  Description of Artificial Sequence Synthetic oligonucleotide
agggtgatctctccaccc

SEQ ID NO: 49 - DNA, 18 bp, Artificial Sequence
  Description of Artificial Sequence Synthetic oligonucleotide
cgcccagggtgatctctc

SEQ ID NO: 50 - DNA, 18 bp, Artificial Sequence
  Description of Artificial Sequence Synthetic oligonucleotide
tgccccgcccagggtgat

SEQ ID NO: 51 - DNA, 18 bp, Artificial Sequence
  Description of Artificial Sequence Synthetic oligonucleotide
agcactgccccgcccagg

SEQ ID NO: 52 - DNA, 15 bp, Artificial Sequence
  Description of Artificial Sequence Synthetic oligonucleotide
atattctacctggtg

SEQ ID NO: 53 - DNA, 15 bp, Artificial Sequence
  Description of Artificial Sequence Synthetic oligonucleotide
catattctacctggt

SEQ ID NO: 54 - DNA, 15 bp, Artificial Sequence
  Description of Artificial Sequence Synthetic oligonucleotide
tcatattctacctgg

SEQ ID NO: 55 - DNA, 15 bp, Artificial Sequence
  Description of Artificial Sequence Synthetic oligonucleotide
atcatattctacctg

SEQ ID NO: 56 - DNA, 15 bp, Artificial Sequence
  Description of Artificial Sequence Synthetic oligonucleotide
gatcatattctacct

SEQ ID NO: 57 - DNA, 15 bp, Artificial Sequence
  Description of Artificial Sequence Synthetic oligonucleotide
tgatcatattctacc

SEQ ID NO: 58 - DNA, 15 bp, Artificial Sequence
  Description of Artificial Sequence Synthetic oligonucleotide
ctgatcatattctac

SEQ ID NO: 59 - DNA, 15 bp, Artificial Sequence
  Description of Artificial Sequence Synthetic oligonucleotide
gctgatcatattcta

SEQ ID NO: 60 - DNA, 15 bp, Artificial Sequence
  Description of Artificial Sequence Synthetic oligonucleotide
agctgatcatattct

SEQ ID NO: 61 - DNA, 12 bp, Artificial Sequence
  Description of Artificial Sequence Synthetic oligonucleotide
atcatattctac

SEQ ID NO: 62 - DNA, 52 bp, Homo sapiens
cgccggggggtcagctgatcatattcyacctggtgcttcagtgggatcagcg

SEQ ID NO: 63 - DNA, 71 bp, Homo sapiens
ggccgctgatcccactgaagcaccaggtggaatatgatcagctgaccccccggcgctccaggtgcagagga

SEQ ID NO: 64 - DNA, 57 bp, Homo sapiens
cccactgaagcaccaggtagaatatgatcagctgaccccccggcgctccaggtgcag

(19) United States
(12) Patent Application Publication
(10) Pub. No.: US 2012/0165389 A1
(43) Pub. Date: Jun. 28, 2012

(54) ANTISENSE OLIGONUCLEOTIDES THAT TARGET A CRYPTIC SPLICE SITE IN USH1C AS A THERAPEUTIC FOR USHER SYNDROME

(76) Inventor: Michelle L. Hastings, Lake Bluff, IL (US)

(21) Appl. No.: 13/277,975

(22) Filed: Oct. 20, 2011

Related U.S. Application Data
(60) Provisional application No. 61/394,973, filed on Oct. 20, 2010.
(60) Provisional application No. 61/481,613, filed on May 2, 2011.

(51) Int. Cl. A61K 31/7088

(52) U.S. Cl. 514/44 A

(57) ABSTRACT

The present invention provides a method for treating Usher's syndrome in a human subject including administering to the human subject an oligonucleotide having 8 to 30 linked nucleosides having a nucleobase sequence comprising a complementary region comprising at least 8 contiguous nucleobases complementary to a target region of equal length within exon 3 of an Usher RNA transcript.

### REFERENCE TO RELATED APPLICATIONS

This application claims priority to U.S. Provisional Patent Application No. 61/394,973 “ANTISENSE OLIGONUCLEOTIDES THAT TARGET A CRYPTIC SPLICE SITE IN USH1C AS A THERAPEUTIC FOR USHER SYNDROME” filed Oct. 20, 2010 and U.S. Provisional Patent Application No. 61/481,613 “ANTISENSE OLIGONUCLEOTIDES THAT TARGET A CRYPTIC SPLICE SITE IN USH1C AS A THERAPEUTIC FOR USHER SYNDROME” filed May 2, 2011, the disclosure of both applications are incorporated herein in their entirety by reference and made a part hereof.

### SEQUENCE LISTING

The instant application contains a Sequence Listing which has been submitted in ASCII format via EFS-Web and is hereby incorporated by reference in its entirety. Said ASCII copy, created on Jan. 6, 2012, is named 11246115.txt and is 89,844 bytes in size.

### FEDERALLY SPONSORED RESEARCH OR DEVELOPMENT

Not Applicable.

### BACKGROUND OF THE INVENTION

1. Technical Field

The present invention provides a therapeutic treatment of Usher syndrome by administering to a person in need thereof an antisense oligonucleotide (ASO) that targets the RNA transcripts of the Ush1c gene to correct defective splicing associated with the disease. More particularly, certain ASOs 8-30 mer in size of the present invention base-pair with regions in exon 3 and intron 2 of the Ush1c gene to correct for loss of gene function due to mutations in the Ush 1c gene.

2. Background Art

Usher syndrome is the leading genetic cause of combined blindness and deafness. Usher syndrome is an autosomal recessive disorder characterized by hearing impairment and retinitis pigmentosa (for review, Keats and Corey, 1999). Usher syndrome is the most common genetic disease that involves both hearing and vision loss. Currently, there is no cure for this debilitating disease that affects approximately 4 in every 100,000 births. There are three types of Usher syndrome that are classified by disease severity. Usher syndrome type 1 (Usher I) is the most severe form and is characterized by severe hearing loss and vestibular dysfunction at birth. Ush1 individuals begin to develop vision problems in early adolescence that progress rapidly to complete blindness. There are five genes that have been associated with Usher I: Ush1C, MYO7A, CDH23, PCDH15 and SANS.

Gene therapy is an attractive approach for Usher syndrome treatment. All types of Usher syndrome appear to be inherited recessively and caused by loss of gene function, suggesting that correction of gene expression would be therapeutic. In addition, because of the early hearing loss, Usher syndrome patients could be treated therapeutically prior to retinal degeneration. Traditional gene therapy approaches based on gene delivery is problematic for many of the Usher genes as they are very large. Therapeutic approaches using small molecules that can directly alter gene expression are attractive possibilities that have been largely undeveloped for Usher syndrome. One reason for the lack of progress in the development of therapeutics for Usher syndrome has been the lack of mouse models that accurately represent the human disease. Prior art mouse models for the disease faithfully manifest the hearing and balance disorders found in Usher syndrome but do not exhibit retinal degeneration.

A mouse model of the present invention for Usher syndrome develops both hearing and visual deficiencies characteristic of Usher syndrome. This mouse model is based on a mutation in the USH1C gene, USH1C216A, that results in the activation of a cryptic 5′ splice site that is used preferentially over the normal 5′ splice site. Splicing from the cryptic site produces a truncated mRNA and protein product. This mouse model provides an ideal tool to investigate therapeutic strategies for Usher syndrome and other diseases associated with mutations in splice sites. The present invention provides ASOs that promote correct splicing of the Ush1c216A gene and restore proper Ush1c expression in vitro and in vivo.

### BRIEF DESCRIPTION OF THE DRAWINGS:

FIG. 1 is a representation of the splicing of an Ush1c gene (SEQ ID NO: 63) which provides a full-length mRNA and a mutant Ush1c216A gene that produces a truncated mRNA;

FIG. 2 is a representation of a cell-free splicing analysis of Ush1c and Ush1c216A exon 3 in HeLa nuclear extract;

FIG. 3 is reverse transcription and polymerase chain reaction (RT-PCR) analysis of the splicing of Ush 1 c exon 3 and cells derived from Usher patients with the Ush1c216A mutation after the cells were treated with control ASO (−) or Ush1c_MO1 and demonstrating that the antisense oligonucleotides targeting Ush1CG216A cryptic splice site redirects splicing to the major splice site that generates mRNA coding for full-length Ush1C (harmonin) protein;

FIG. 4 is RT-PCR analysis of the splicing of a Ush1c exon 3 in kidney tissue of Ush1C216A mice injected with Ush1c_MO1 to redirect splicing to the splice site that generates mRNA coding for the full-length protein;

FIG. 5 is a schematic of the Ush1c.216a plasmid and splicing of the transcripts from the minigene following treatment with ASOs;

FIG. 6 summarizes the results of RT-PCR of four 2′ MOE oligonucleotides;

FIG. 7 shows sequence and USH1C target region (SEQ ID NO: 64) of ASO 2′MOE-29 (Sequence ID No. 33). FIG. 7 also discloses sequences for 2′1140E-49, 2′MOE-48 and 2′MOE-28 as SEQ ID NOS 59, 53 and 32, respectively.

### DETAILED DESCRIPTION OF THE INVENTION:

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention provides therapeutic treatment of Usher syndrome by administering an effective amount of an antisense oligonucleotide (ASO) to Usher patients with the Ush1C216A mutation. A recently developed mouse model (Lentz et al., 2006) for Usher syndrome based on an Acadian Usher mutation in Ush1c gene, harmonin has been used to develop a therapeutic treatment for human patients. As used herein, “Ush1c gene” means a gene described in Lentz, J, Pan, F, Ng, S S, Deininger, P, Keats, B. 2007. Ush1c216A knock-in mouse survives Katrina. Mutat. Res. 616: 139-144 and having a sequence [ENSG00000006611Accession number] provided herein as SEQ ID NO. 1, or a variant thereof. In certain embodiments, an Usher gene is at least 90% identical to Accession Number ENSG00000006611, set forth as SEQ ID NO 1.

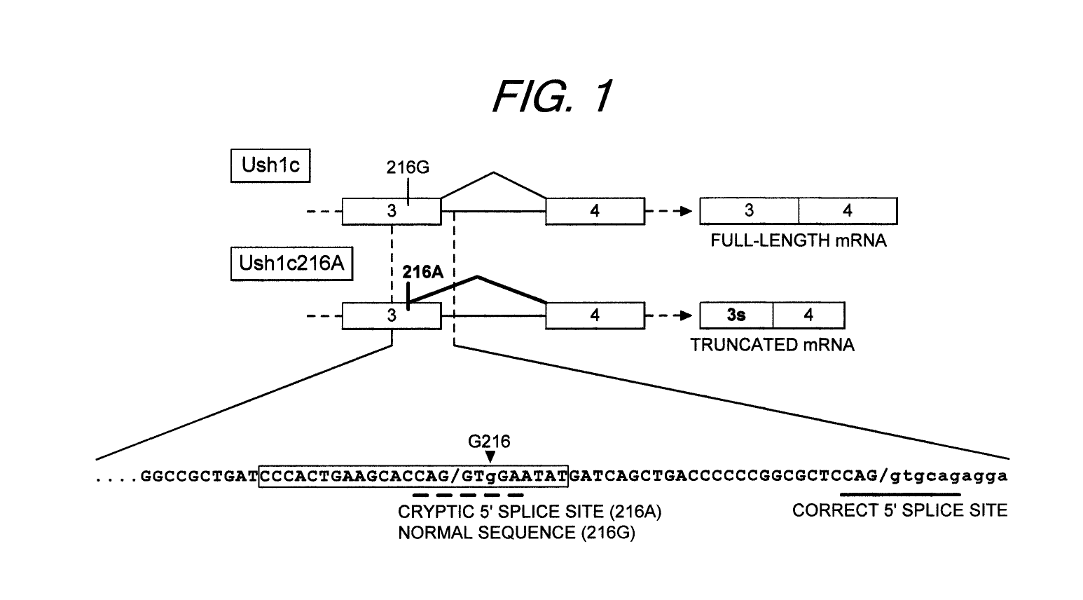

FIG. 1 shows the Ush1c216A mutation is located in exon 3 of the gene and creates a cryptic 5′ splice site which is used preferentially over the correct splice site (Bitner-Glindzicz et al., 2000; Verpy et al., 2000; Lentz et al., 2004). The resulting mRNA is out of frame and codes for a truncated protein product. The Ush1c216A mouse has the 216A mutation knocked into the mouse Ush1c gene. Mice homozygous for the Ush1c216A mutation exhibit classic circling behavior indicative of severe vestibular dysfunction and deafness. The mice also show evidence of retinal degeneration.

### Pre-mRNA Splicing

Pre-mRNA splicing involves the precise and accurate removal of introns from the pre-messenger RNA and the ligation of exons together after intron removal to generate the mature mRNA which serves as the template for protein translation. Pre-mRNA splicing is a two-step reaction carried out by a spliceosome complex comprising protein and small RNA components which recognize conserved sequence elements within the introns and exons of the RNA. Recognition of these sequence elements, including the 5′ splice site, 3′ splice site and branch point sequence, is the primary mechanism directing the correct removal of introns.

Splicing requires direct base-pairing between small nuclear RNA (snRNA) components of the spliceosome and the splice site nucleotides of the mRNA. This interaction can be easily disrupted by gene mutations or by artificial blocking using short oligonucleotides complementary to the RNA. Such so called antisense oligonucleotides (ASOs), when designed to be complementary to a splice sites, will compete for base-pairing with the snRNAs, thereby blocking an essential step in splicing at the site. In this way, antisense oligonucleotides can potently block unwanted splicing or redirect splicing to alternative splice sites.

### Therapeutic Perspectives

Mutations that alter pre-mRNA splicing are found in more than 50% of genes associated with deafness. Developing methods to manipulate splicing will benefit the development of therapies for all disease-associated mutations that affect splicing. Although disease-causing mutations that disrupt splicing are common, there are relatively few tools available to study these types of defects in vivo. Only a handful of animal models for disease have been developed that are based on splicing mutations. Animal models for SMA that reproduce the exact splicing defect in SMA in humans have been instrumental in the forward progress that has been made in developing potential therapeutics for the disease (Hua et al., 2010). Many of these therapies are based on either small molecule compounds or ASOs that alter the splicing pattern of the pre-mRNA (Sumner 2006).

ASOs have been effectively used to alter pre-mRNA splicing (for review, Aartsma-Rus & van Ommen 2007; Smith et al., 2006). ASOs targeted to cryptic splice sites created by mutations in the ATM gene were recently demonstrated to effectively redirect splicing to the correct splice site and improve protein expression (Du et al., 2007). The first clinical trials based on ASO-induced skipping of exons as a therapy for Duchenne muscular dystrophy (DMD) have shown success in increasing dystrophin protein levels in muscle cells surrounding the site of injection (van Deutekom et al., 2008). ASO-based therapies may provide a customizable approach to mutation-based treatments for disease. The effectiveness of ASOs in modulating splicing in a therapeutically beneficial manner has been demonstrated for a number of diseases.

One preferred form of the invention provides a therapeutic treatment of human subjects having Usher syndrome by administering to the human subject an ASO oligonucleotide having 8 to 30 linked nucleosides having a nucleobase sequence comprising a complementary region comprising at least 8 contiguous nucleobases complementary to a target region of equal length within exon 3 of an Usher transcript.

In a preferred form of the invention, suitable ASOs when administered to a patient in need thereof will promote the correct splicing of the USH1C216A transcript to provide an mRNA which serves as the template for transcribing the full-length, harmonin protein. More preferably, suitable ASOs will complementary base pair to an effective number of nucleotides of exon 3 of the pre-mRNA transcript of the USH1C216A mutation to redirect the splicing from the cryptic 5′ splice site to the major 5′ splice site. In a more preferred form of the invention, suitable ASOs will complementary base pair to consecutive nucleotides of exon 3 and have a length of 8 to 30 mer, more preferably 15 to 30 mer, even more preferably 15 to 27 mer and most preferably 15-25 mer or any range or combination of ranges therein.

Suitable ASOs can be chemically modified to be different from their natural nucleic acid structure to prevent enzymatic degradation, triggering of the innate immune response or inflammation response. Chemical modifications can be nucleoside modification (i.e., to the sugar moiety and or to the nucleobase moiety) and/or modifications to internucleoside linkages. In one preferred form of the invention, suitable ASOs have their nucleic acid bases bound to a morpholine ring instead of a ribose ring and are linked through a non-ionic phosphorodiamidate groups instead of an anionic phosphodiester group. These modified oligonucleotides are available from Gene Tools under the tradename MORPHOLINO.

Other suitable modifications include replacing the ribose rings with furanosyl or substituted furanosyl rings where the substituents, in some instances but not necessarily, form bridges within the furanosyl ring to form bicyclic sugars or bridges to other ring structures to form tricyclic sugars. Nucleosides that contain bicyclic and tricylic sugar moieties shall be referred to respectively as bicyclic nucleosides and tricyclic nucelosides and those that contain a single ring may be referred to as monocyclic. It is also contemplated replacing the oxygen atom in the furanosyl with a non-oxygen atom such as carbon, sulfur or nitrogen. In a more preferred form of the invention, the furanosyl 2′-position will have a 2-methoxy ethyl ether substituent with the following structure —OCH₂CH₂OCH₃ (“2′-MOE”). Suitable chemically modified ASOs are available from Isis Pharmaceuticals, Inc.

It is also contemplated that the ASOs may have conjugate groups attached thereto, as is well known in the art, to provide a desired property or characteristic such as pharmacodynamics, pharmacokinetics, stability, targeting, binding, absorption, cellular distribution, cellular uptake, charge and clearance.

The present invention further provides therapeutic dosage forms for delivery to a human subject. It is contemplated that the ASOs described herein can be delivered by any suitable route of administration including parenteral, oral, injection, transdermal, intramuscular, topical, or other route of administration known to those skilled in the art. In a most preferred form of the invention the ASO is injected directly into the eye or ear or both of the human subject.

### MORPHOLINO OLIGONUCLEOTIDES EXAMPLES

### Example 1

### Development of an Ush1c216A Splicing System to Test ASOs and Small Molecules

The present invention provides an Ush1c and Ush1c216A minigene comprising exon 3, intron 3 and exon 4 of the Ush1c gene. These minigenes are used as templates to create wild-type and G216A mutant Ush1c mRNA that can be spliced in HeLa nuclear extract. The splicing of these transcripts in HeLa nuclear extract results in faithful recapitulation of the expected full-length splicing of the wild-type gene and cryptic splicing from the G216A mutated transcript. These results demonstrate that this cell-free system can be used to accurately model normal and disease-associated splicing caused by the G216A mutation.

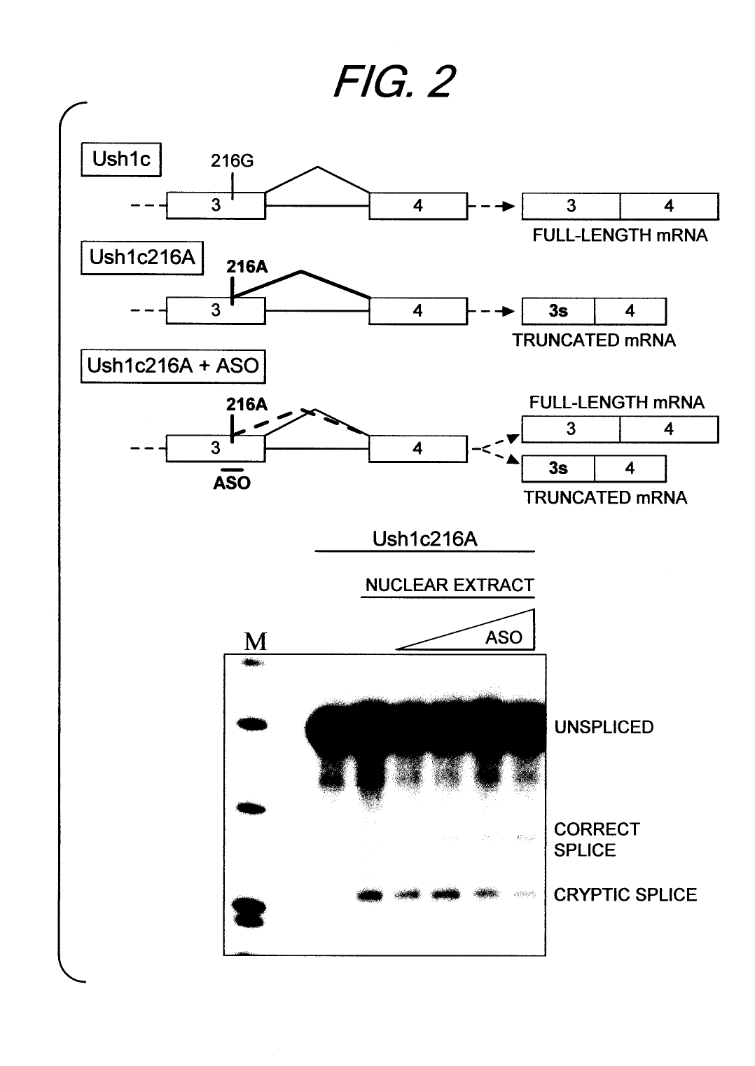

We next tested several ASOs targeted to the cryptic 5′ splice site in the cell-free splicing system and assessed switching from the use of the cryptic 5′ splice site to the correct 5′ splice site. FIG. 2 shows these ASOs effectively increased splicing to the correct 5′ splice site in a dose-dependent manner. These results demonstrate the utility of the cell-free splicing system for testing ASOs and the ability to modulate the use of the cryptic and normal 5′ splice site using ASOs.

### Example 2

### ASOs That Improve Ush1c216A Slicing in Cell Culture

The effectiveness of ASOs in achieving splice-site switching in cultured cells was tested. An Ush1c minigene expression system was created to test the effect of the ASOs on the splicing mutant Ush1c gene transcripts in cells. The ASOs effectively correct the defective splicing and result in the generation of normally spliced mRNA.

We have also developed cell lines from the tissues of Ush1C216A mice that carry the human mutation that creates the cryptic splice sites. The ASOs potently redirect splicing to the correct splice site thereby rescuing Ush1c expression.

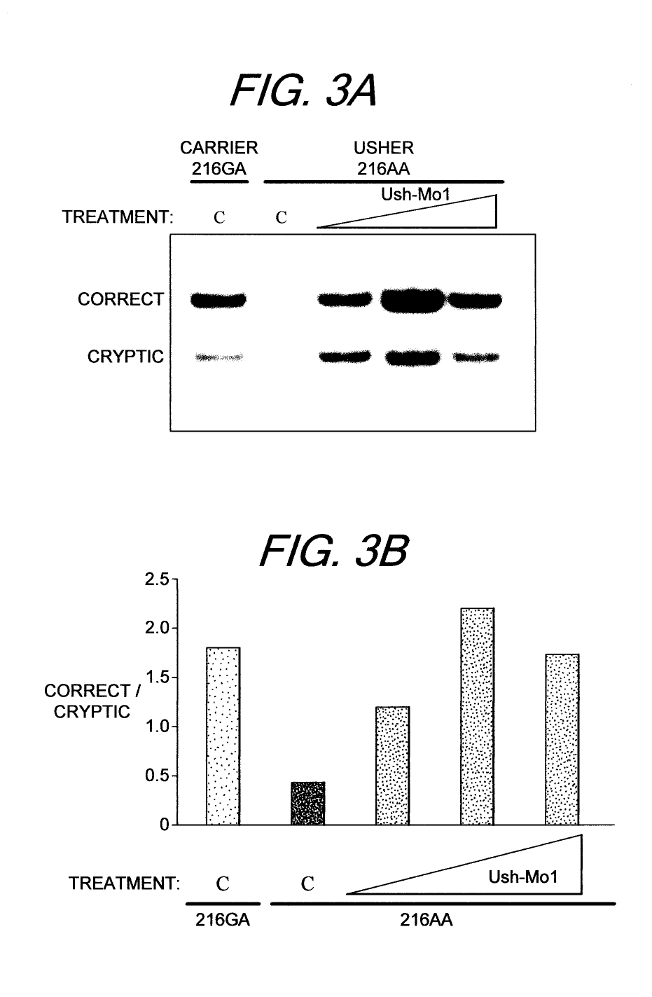

FIG. 3 shows that we have successfully corrected splicing of Ush1C216A mRNA arising from the human Ush1C216A gene in cell lines derived from a patient with Usher Syndrome carrying the Ush1C216A mutation in the Ush1C gene.

### Example 3

### Correction of Ush1c216A Exon 3 Cryptic Splicing in Mice Using Optimized ASOs

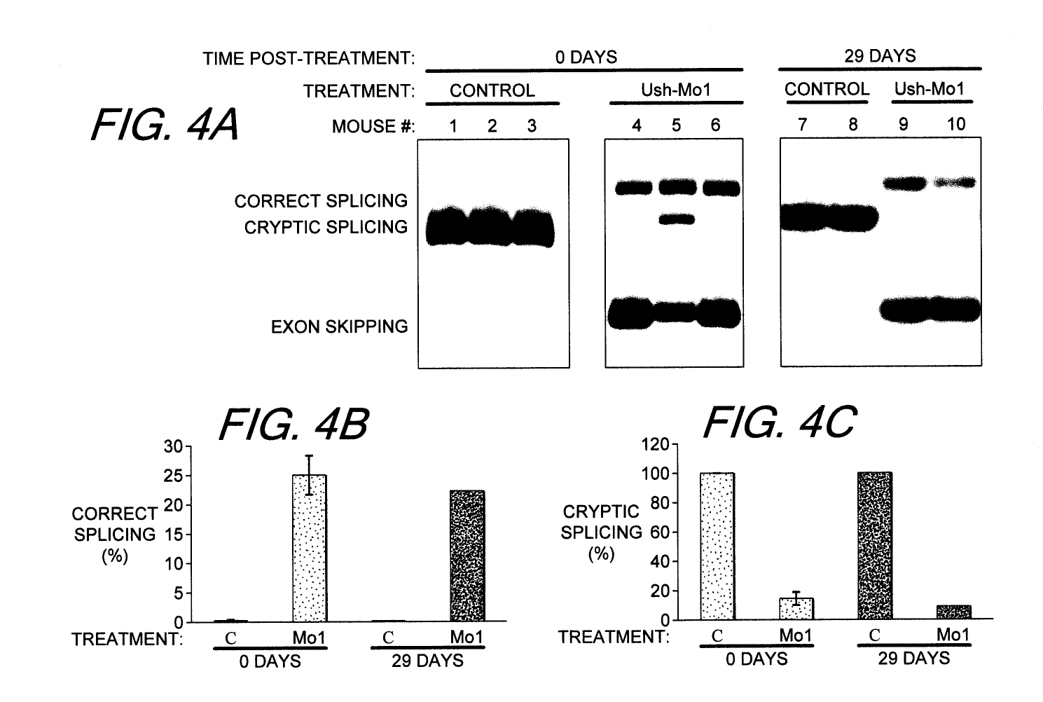

The ASOs that we have utilized shown in Table 1,2 to target cryptic splicing in Usher syndrome shown herein have been tested in an Ush1c.216a minigene expression system (Table 1) and in the Ush1c216A knock-in Usher syndrome mouse model (Table 2). FIG. 4 shows that the preliminary results indicate that the ASOs correct splicing in the cells of a number of tissues such as the kidney, and that this effect can last for at least 29 days after the final treatment.

TABLE 1
Modulation of Ush1c.216A splicing of RNA transcripts
from a Ushlc.216A minigene.

| MORPHOLINO | Start | | | % cryptic | SEQ |
|---|---|---|---|---|---|
| NO | Site | Sequence | Region | splicing | ID NO |
| Ush1C_MO1 | 138577 | AGCTGATCATATTCTACCTGGTGCT | USH1C | 2.84 | 2 |
| | | | Exon 3 | | |
| | | | (G to A | | |
| | | | mt) | | |
| Ush1C_MO2 | 138569 | ATATTCCACCTGGTGCTTCAGTGGG | USH1C | 5.75 | 3 |
| | | | exon 3 | | |
| | | | (G/A | | |
| | | | mt) | | |

TABLE 2
Modulation of Ush1.c.216A splicing in mice kidney using vivo-morpholinos

| MORPHOLINO | Start | | | % cryptic | SEQ |
|---|---|---|---|---|---|
| NO | Site | Sequence | Region | splicing | ID NO |
| N/A | N/A | N/A | control | 99.543 | N/A |
| | | | treated | | |
| Ush1C_MO1 | 138577 | AGCTGATCATATTCTACCTGGTGCT | USH1C | 11.45 | 4 |
| | | | exon 3 | | |
| | | | (G to A | | |
| | | | mt) | | |

### ISIS PHARMACEUTICAL 2′-MOE EXAMPLES

### Example 4

### Ush1c.216a Minigene

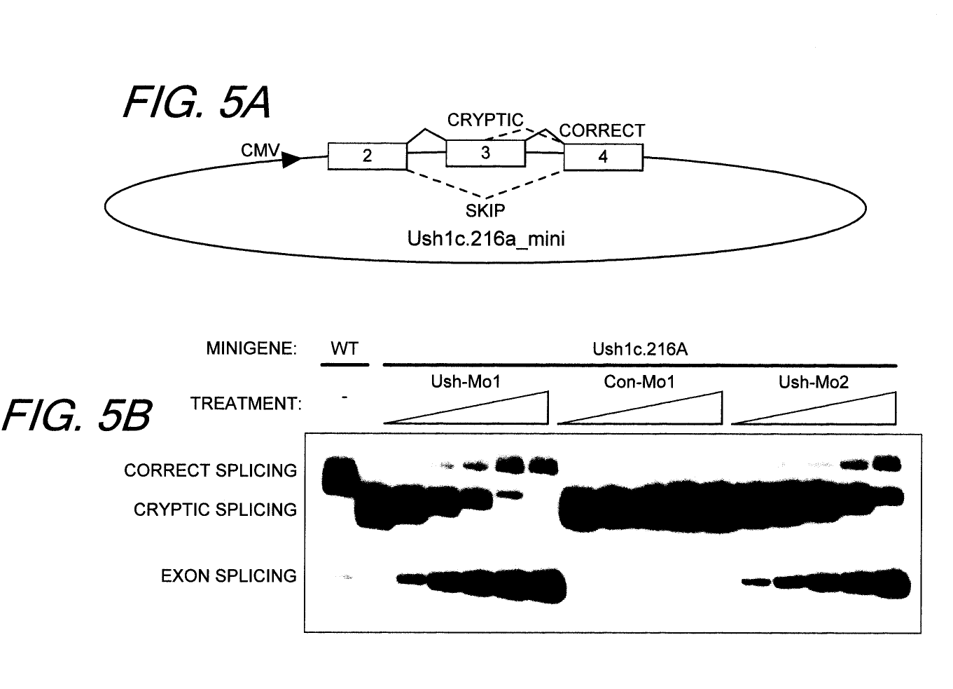

A plasmid comprising an Usher 1C minigene having a 216A mutation (Ush1c.216a) was prepared using standard molecular biology techniques. The Ush1c.216a plasmid included exons 2, 3, and 4, and introns 2 and 3. The minigene was under control of the CMV promoter. A schematic of the Ush1c.216a plasmid appears in FIG. 5.

### Example 5

### Antisense Modulation of Usher RNA Transcript Splicing

Antisense oligonucleotides complementary to different regions of the Usher transcript were tested for their ability to modulate splicing of RNA transcripts expressed from the Usher minigene. Antisense oligonucleotides comprising 2′MOE modified nucleosides (Tables 3,4) in which each nucleoside of the oligonucleotides was a 2′-MOE modified nucleoside and intemucleoside linkages were phosphorothioate linkages. All of the nucleobases were unmodified and cytosine bases were 5-meC.

To test the ability of the antisense oligonucleotides to modulate Usher transcript splicing, HeLa cells were co-transfected with the Ush1c.216a plasmid from Example 4 and an antisense oligonucleotide (or no antisense oligonucleotide in the case of the untreated control). The results are summarized in Tables 3,4). The start site is the position relative to 13475 of SEQ ID NO 1.

TABLE 3
Modulation of USH1C pre-mRNA splicing by Isis 18 nucleotide 2′-MOE
modified oligonucleotides shown 5′ to 3′ direction.

| ISIS | Start | | | % cryptic | SEQ |
|---|---|---|---|---|---|
| NO | Site | Sequence | Region | splicing | ID NO |
| N/A | N/A | N/A | Untreated | 100 | N/A |
| | | | Control | | |
| 527106 | 138475 | ACGGCCACGTCCATGGTC | USH1C | 13.39 | 5 |
| | | | exon 3 | | |
| 527107 | 138480 | CGAGCACGGCCACGTCCA | USH1C | 6.29 | 6 |
| | | | exon 3 | | |
| 527108 | 138485 | TCCCACGAGCACGGCCAC | USH1C | 9.93 | 7 |
| | | | exon 3 | | |
| 527109 | 138490 | AGGTCTCCCACGAGCACG | USH1C | 32.49 | 8 |
| | | | exon 3 | | |
| 527110 | 138495 | GCTTCAGGTCTCCCACGA | USH1C | 34.79 | 9 |
| | | | exon 3 | | |
| 527111 | 138500 | GACCAGCTTCAGGTCTCC | USH1C | 64.21 | 10 |
| | | | exon 3 | | |
| 527112 | 138505 | TTGATGACCAGCTTCAGG | USH1C | 23.89 | 11 |
| | | | exon 3 | | |
| 527113 | 138510 | GTTCATTGATGACCAGCT | USH1C | 34.68 | 12 |
| | | | exon 3 | | |
| 527114 | 138515 | GCTGGGTTCATTGATGAC | USH1C | 41.71 | 13 |
| | | | exon 3 | | |
| 527115 | 138520 | AGACGGCTGGGTTCATTG | USH1C | 12.15 | 14 |
| | | | exon 3 | | |
| 527116 | 138525 | GAGGCAGACGGCTGGGTT | USH1C | 36.97 | 15 |
| | | | exon 3 | | |
| 527117 | 138530 | AAACAGAGGCAGACGGCT | USH1C | 26.32 | 16 |
| | | | exon 3 | | |
| 527118 | 138535 | GCATCAAACAGAGGCAGA | USH1C | 22.23 | 17 |
| | | | exon 3 | | |
| 527119 | 138540 | GAATGGCATCAAACAGAG | USH1C | 29.63 | 18 |
| | | | exon 3 | | |
| 527120 | 138545 | CGGCCGAATGGCATCAAA | USH1C | 63.65 | 19 |
| | | | exon 3 | | |
| 527121 | 138550 | ATCAGCGGCCGAATGGCA | USH1C | 15.79 | 20 |
| | | | exon 3 | | |
| 527122 | 138555 | GTGGGATCAGCGGCCGAA | USH1C | 57.54 | 21 |
| | | | exon 3 | | |
| 527123 | 138560 | CTTCAGTGGGATCAGCGG | USH1C | 5.27 | 22 |
| | | | exon 3 | | |
| 527124 | 138563 | TGCTTCAGTGGGATCAGC | USH1C | 3.61 | 23 |
| | | | exon 3 | | |
| 527125 | 138566 | TGGTGCTTCAGTGGGATC | USH1C | 9.68 | 24 |
| | | | exon 3 | | |
| 527126 | 138569 | ACCTGGTGCTTCAGTGGG | USH1C | 21.75 | 25 |
| | | | exon 3 | | |
| 527127 | 138569 | ATATTCTACCTGGTGCTTCAGTGGG | USH1C | 16.77 | 26 |
| | | | exon 3 | | |
| | | | (G to A | | |
| | | | mt) | | |
| 527128 | 138571 | CTACCTGGTGCTTCAGTG | USH1C | 22.39 | 27 |
| | | | exon 3 | | |
| | | | (G to A | | |
| | | | mt) | | |
| 527129 | 138573 | TTCTACCTGGTGCTTCAG | USH1C | 24.45 | 28 |
| | | | exon 3 | | |
| | | | (G to A | | |
| | | | mt) | | |
| 527130 | 138576 | ATATTCTACCTGGTGCTT | USH1C | 14.89 | 29 |
| | | | exon 3 | | |
| | | | (G to A | | |
| | | | mt) | | |
| 527131 | 138577 | AGCTGATCATATTCTACCTGGTGCT | USH1C | 2.35 | 30 |
| | | | exon 3 | | |
| | | | (G to A | | |
| | | | mt) | | |
| 527132 | 138579 | ATCATATTCTACCTGGTG | USH1C | 12.13 | 31 |
| | | | exon 3 | | |
| | | | (G to A | | |
| | | | mt) | | |
| 527133 | 138581 | TGATCATATTCTACCTGG | USH1C | 2.85 | 32 |
| | | | exon 3 | | |
| | | | (G to A | | |
| | | | mt) | | |
| 527134 | 138584 | AGCTGATCATATTCTACC | USH1C | 2.70 | 33 |
| | | | exon 3 | | |
| | | | (G to A | | |
| | | | mt) | | |
| 527135 | 138586 | TCAGCTGATCATATTCTA | USH1C | 19.98 | 34 |
| | | | exon 3 | | |
| | | | (G to A | | |
| | | | mt) | | |
| 527136 | 138589 | GGGTCAGCTGATCATATT | USH1C | 98.82 | 35 |
| | | | exon 3 | | |
| 527137 | 138591 | GGGGGTCAGCTGATCATA | USH1C | 99.28 | 36 |
| | | | exon 3 | | |
| 527138 | 138593 | CGCCGGGGGGTCAGCTGA | USH1C | 99.60 | 37 |
| | | | exon 3 | | |
| 527139 | 138598 | TGGAGCGCCGGGGGGTCA | USH1C | 90.93 | 38 |
| | | | exon 3 | | |
| 527140 | 138603 | GCACCTGGAGCGCCGGGG | USH1C | 97.59 | 39 |
| | | | exon | | |
| | | | 3/intron3 | | |
| 527141 | 138608 | CCTCTGCACCTGGAGCGC | USH1C | 99.81 | 40 |
| | | | exon | | |
| | | | 3/intron3 | | |
| 527142 | 138613 | GGCTTCCTCTGCACCTGG | USH1C | 99.54 | 41 |
| | | | exon | | |
| | | | 3/intron3 | | |
| 527143 | 138618 | CTGGTGGCTTCCTCTGCA | USH1C | 97.64 | 42 |
| | | | intron 3 | | |
| 527144 | 138623 | CCAGCCTGGTGGCTTCCT | USH1C | 96.34 | 43 |
| | | | intron 3 | | |
| 527145 | 138628 | TGCCTCCAGCCTGGTGGC | USH1C | 94.86 | 44 |
| | | | intron 3 | | |
| 527146 | 138633 | CCCCCTGCCTCCAGCCTG | USH1C | 96.78 | 45 |
| | | | intron 3 | | |
| 527147 | 138638 | CTCCACCCCCTGCCTCCA | USH1C | 98.2 | 46 |
| | | | intron 3 | | |
| 527148 | 138643 | GATCTCTCCACCCCCTGC | USH1C | 97.94 | 47 |
| | | | intron 3 | | |
| 527149 | 138648 | AGGGTGATCTCTCCACCC | USH1C | 97.82 | 48 |
| | | | intron 3 | | |
| 527150 | 138653 | CGCCCAGGGTGATCTCTC | USH1C | 94.03 | 49 |
| | | | intron 3 | | |
| 527151 | 138658 | TGCCCCGCCCAGGGTGAT | USH1C | 97.74 | 50 |
| | | | intron 3 | | |
| 527152 | 138663 | AGCACTGCCCCGCCCAGG | USH1C | 97.83 | 51 |
| | | | intron 3 | | |

TABLE 4
Modulation of USH1C pre-mRNA splicing by Isis 2′-MOE modified 15
nucleotide oligonucleotides shown in 5′ to 3′ direction.

| | | | | % | |
|---|---|---|---|---|---|
| ISIS | Start | | | cryptic | SEQ |
| NO | Site | Sequence | Target | splicing | ID NO |
| 535400 | 138579 | ATATTCTACCTGGTG | USH1C exon | 53.03 | 52 |
| | | | 3 (G to A mt) | | |
| 535401 | 138580 | CATATTCTACCTGGT | USH1C exon | 61.03 | 53 |
| | | | 3 (G to A mt) | | |
| 535402 | 138581 | TCATATTCTACCTGG | USH1C exon | 66.12 | 54 |
| | | | 3 (G to A mt) | | |
| 535403 | 138582 | ATCATATTCTACCTG | USH1C exon | 41.61 | 55 |
| | | | 3 (G to A mt) | | |
| 535404 | 138583 | GATCATATTCTACCT | USH1C exon | 22.64 | 56 |
| | | | 3 (G to A mt) | | |
| 535405 | 138584 | TGATCATATTCTACC | USH1C exon | 27.35 | 57 |
| | | | 3 (G to A mt) | | |
| 535406 | 138585 | CTGATCATATTCTAC | USH1C exon | 20.08 | 58 |
| | | | 3 (G to A mt) | | |
| 535407 | 138586 | GCTGATCATATTCTA | USH1C exon | 16.79 | 59 |
| | | | 3 (G to A mt) | | |
| 535408 | 138587 | AGCTGATCATATTCT | USH1C exon | 72.49 | 60 |
| | | | 3 (G to A mt) | | |
| 535409 | 138588 | ATCATATTCTAC | USH1C exon | 98.38 | 61 |
| | | | 3 (G to A mt) | | |

### Example 6

### Antisense Modulation of Usher Transcript

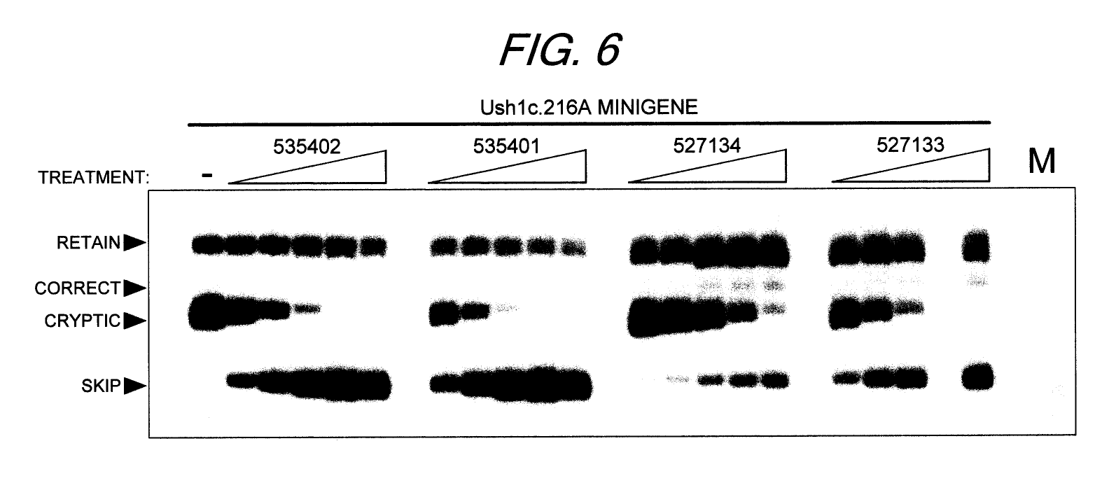

Four of the antisense oligonucleotides above were separately tested at varying doses (0 (control), 5 nM, 10 nM, 20 nM, 40 nM, and 80 nM). Each antisense oligonucleotide reduced the amount of cryptic spliced transcript and increased the amount of correctly spliced or exon 3-skipped transcript in a dose-dependent manner. RNA was collected and analyzed by RT-PCR. Results are summarized in FIG. 6.

### Example 7

### In Vivo Modulation of the Usher Transcript

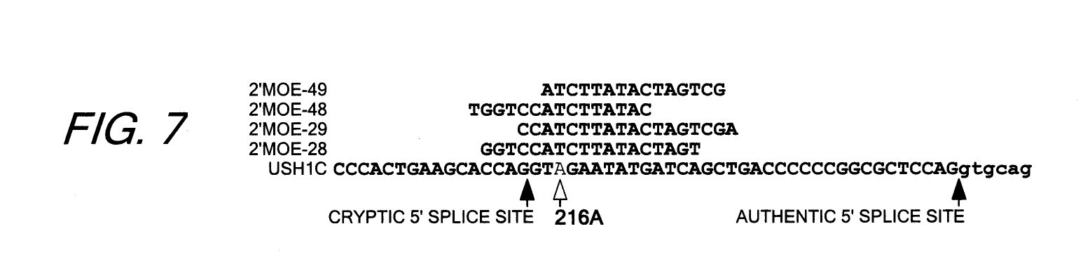

Mice having the 216A mutation in their Ush1c gene have been described. Such mice have congenital hearing loss and retinal degeneration. Four of the above described antisense oligonucleotides are shown in their 3′ to 5′ direction in FIGS. 7 (527133, 527134, 535401, and 535407 (Sequence ID Nos. 32, 33, 53 and 59 respectively)) were administered to such mice to test their ability to modulate splicing in vivo.

Doses of 50mg/kg were administered by intraparitoneal injection twice each week for two weeks. Two days after the final injection, the mice were euthanized and RNA was isolated from various tissues. RNA was analyzed by radiolabeled RT-PCR. Splicing modulation was detected in the tissues of treated mice.

### Example 8

### Correction of Hearing and Vestibular Dysfunction in a Mouse Model for Deafness

Hearing defects are present in approximately 1 in 500 newborns, and in developed countries, frequently result from single locus gene mutations^{1,2}. Here, we use a mouse model of congenital, inherited deafness to investigate a potential cure for hearing loss and vestibular dysfunction using an antisense oligonucleotide splice targeting approach. Mice homozygous for the Ush1c.216A mutation (216AA), which causes Usher syndrome in humans, exhibit circling behavior indicative of severe vestibular dysfunction and deafness³. ASOs were designed to specifically redirect splicing of USH1C 216A RNA transcripts from a cryptic splice site, which is activated by the mutation, to the authentic site. ASOs were optimized in cell-free and cellular assays and are shown to correct splicing of the disease 216A RNA in an Usher syndrome patient cell line. A single treatment of ASOs in 216AA neonate mice corrects splicing in the cochlea, eliminates vestibular dysfunction and restores hearing to a level comparable to wild-type mice. Our results indicate a cure for deafness and vestibular dysfunction in mice using ASOs, demonstrating that hearing can be treated by correction of gene expression at an early stage in development.

To identify ASOs that can block splicing at the cryptic splice site created by the 216A mutation, we constructed an USH1c minigene (FIG. 5) comprising exons 2-4 and the intervening introns of human USH1C 216G (WT) or 216A cloned into an expression plasmid. The minigene plasmids and ASOs with 2′-O-methoxyethyl (2′-MOE) sugar modifications and a phosphodiester backbone were transfected into cells and splicing was analyzed after 48 hours by radiolabeled, reverse-transcription PCR (RT-PCR) analysis of isolated RNA. Forty-seven 2′-MOE 18-mer ASOs complementary to regions in exon 3 and the 5′ end of intron 3 as set forth in Table 3 above were tested and ten 2′-MOE 15-mer ASOs as shown in Table 4. The ASOs start with the first position of exon 3, with overlapping ASOs providing coverage in 5-nucleotide increments. The premise of these experiments is that there may be exonic splicing enhancers or silencers that could be targeted to modulate splicing of the cryptic or correct splice site. ASOs targeted to the region surrounding the 216A mutation strongly blocked cryptic splicing and promoted correct splicing. Many of the ASOs-targeted to regions throughout the exon also caused skipping of exon 3. The mRNA lacking exon 3 encodes a full-length protein lacking 48 amino acids flanking the N-terminus of the first PDZ domain of the protein. ASOs identified as 2′MOE 28 and 29 in FIG. 1c correspond to Isis Nos. 527133 and 527134 (Sequence ID Nos. 32 and 33) in Table 3 were most effective at correcting splicing and blocking cryptic splicing.

Optimal ASO concentrations for blocking cryptic splicing and restoring correct splicing was tested using the USH1C minigene expression system described above and treating cells with increasing concentrations of 2′MOEs that were most effective in the ASO walk experiments (2′MOE-28, 29 or Sequence ID Nos. 32 and 33) along with shorter versions of these ASOs (2′MOE-48,49, Isis Nos. 535401 and 535407, Sequence ID Nos. 53 and 59 respectively in Table 4) (FIG. 7). All of the 2′MOE ASOs blocked cryptic, with cryptic splicing nearly abolished in samples treated with 12 μM ASO (FIG. 1d).

To test the effect of ASOs in vivo, adult Ush1c.216AA mice were injected with 50 mg/kg of 2′MOE-28, 29, 48 or 49 (Sequence ID Nos. 32, 33, 53 and 59) twice a week for two weeks for a total of four injections and kidneys were collected 24 hours after the final injection. 2′MOE-29 corrected splicing of 216AA in the kidney of treated mice. Optimal dosing was determined by injecting mice with different amounts of 2′MOE-29 using the dosing regimen described above. 2′MOE-29 corrected splicing and increased harmonin protein expression in a dose-dependent manner. No change in behavior was evident in the adult mice following ASO injection.

Harmonin is first expressed between embryonic day 15 and postnatal day 15 (P15)⁴, during the time when hearing is being established suggesting that neonate expression of harmonin may be critical for hearing development. Thus, we treated neonatal mice and tested the ability of the ASOs to correct vestibular and hearing defects. Mice were treated at P3, P5, P10 or P16 by intraperitoneal injection of 2′MOE-29 (Sequence ID No. 33). Untreated mice or those treated with a mismatched 2′MOE (2′MOE-C) ASO displayed general hyperactivity and circling behavior characteristic of the vestibular defects and deafness by postnatal day 21 as previously reported⁵. In contrast, the behavioral activity of mice treated with 2′MOE-29 (Sequence ID No. 33) was indistinguishable from heterozygote 216GA or wildtype 216GG mice, with no circling, head-tossing or hyperactivity. There was no discernable difference between mice treated at P3, P5 or P10, whereas P16-treated mice were indistinguishable from untreated mutant 216AA mice. The oldest P5 2′MOE-29-treated mice are now 6 months of age and do not exhibit hyperactivity or circling behavior, suggesting that the ASOs can effectively treat the vestibular dysfunction associated with Usher syndrome when delivered early in neonate development.

To assess hearing function, auditory-evoked brainstem response (ABR) analysis was performed. ABR thresholds to broad-band (BB) and pure tone stimuli (8, 16 and 32 kHz) were compared in one month old 216AA mutant mice treated with 2′MOE-29 (Sequence ID No. 33) with those of age-matched control mice. The following control mice were used: treated and untreated wild type (wt, 216GG) and heterozygote (het, 216GA) mice (referred to as wt/het ctl); and untreated mutants and mutants treated with 2′MOE-C (mut 2′MOE-C). Wt and het littermates had the expected thresholds of mice with normal hearing, and there was no difference with treatment (2′MOE-29 (SEQ. ID No. 33) or 2′MOE-C). Untreated mutants (216AA) and mutants treated with the mismatched 2′MOE-C had an abnormal (fewer peaks or greater interpeak latency) or no response at 90 dB SPL to BB or pure tones. In contrast, 216AA mutant mice treated between P3-5 with a single dose of 2′MOE-29 (SEQ. ID No. 33) had normal audiograms with the expected 4-5 peaks and normal thresholds to BB and 8 and 16 kHz pure tones comparable to wt/het control mice, (48 (BB), 46 (8 kHz), 47 (16 kHz) dB SPL 216AA 2′MOE-29, n=12; 37 (BB), 39 (8 kHz), 38 (16 kHz) dB SPL wt/het ctl, n=16). Thresholds to 32 kHz in 2′MOE-29-treated mutants were slightly lower (88 dB SPL, n=12) than control mutants (>90 dB SPL, n=11), however were considerably higher than wt/het ctl thresholds (51 dB SPL wt/het ctl, n=16). These data show rescue of low and mid frequency hearing and to a lesser degree high frequency. 216AA mutant mice treated with a single dose of 2′MOE-29 (SEQ. ID No. 33) at P10 had more variable responses with higher thresholds than those treated at P4-5 (78 (BB), 72 (8 kHz), 73 (16 kHz), >90 (32 kHz) dB SPL, n=5), but lower than untreated mutants or mutants treated with 2′MOE-C, indicating a developmental window of therapeutic efficacy in mice.

ABRs were also performed at 2 and 3 months of age to determine the duration of auditory rescue. These results show that the mice injected between P3 and P5 of age and to a lesser extent at P10, can hear at 1, 2 and 3 months of age, indicating an effective correction of deafness with a single ASO-treatment early in life.

Cochleae from mice injected at P5 with 2′MOE-Ush-29 (SEQ. ID No. 33) or 2′MOE-mis, were harvested at 1 month of age and subjected to RT-PCR and western blot analyses. A low level of correct exon 3 splicing was observed in the 2′MOE-29-treated 216AA mice that was not seen in the control treated mice. The correction was not at the level of correct splicing observed in unaffected 216GA mice. It is likely that the extent of splicing correction was greater immediately after treatment when the ASO would have been at the highest concentration during a critical time-period for cochlear and vestibular hair cell development. Harmonin protein levels in cochleae isolated from 2′MOE-Ush-treated mice were higher than that from mice treated with 2′MOE-mis mice and similar to protein levels of 216GA mice.

Cochleae were also microdissected harvest organs or corti and subjected to immunohistochemistry. The microdissected organs of corti labeled with DAPI (blue), parvalbumin (red), and neurofilament (green) show the physical structure of the cochleae were consistent with wt/het control mice.

Discussion

Our results strongly suggest that we have cured deafness in Usher syndrome using a single injection of ASO shortly after birth. This indicates that genetic forms of deafness can be effectively treated and that this treatment may only need to occur once in life, during the critical hair cell developmental period.

The correction of hearing in Usher syndrome demonstrates that deafness can be treated if interventions occur at an early time point in development. In mice, our results show that treatment at P10 leads to correction of vestibular dysfunction and partial restoration of hearing, whereas treatment at P3-P5 results in mice that have no vestibular deficits and have ABRs that are nearly identical to wild-type mice. Although harmonin is expressed as early as E15 in mice⁴ our results suggest that expression between E15 and P5 is not required for the development of low and mid-frequency hearing. The only quantifiable difference in 216AA mutant mice treated with Ush-2′MOE-29 and 216GA or GG mice is hearing at high frequencies (32 kHz, FIG. 9b). Because detection of high frequency sound occurs at the base of the cochlea, this result may suggest that Ush1c is expressed tonotopically during development, and when treated at P3-5, splicing is only corrected in the mid-apical regions of the cochlea.

Individuals affected with Usher syndrome suffer a tremendous burden from the dual sensory loss of hearing and vision, and the correction of one of these sensory deficits will have a significant positive impact. The retinitis pigmentosa associated with Usher syndrome is recapitulated in the Ush1c.216AA mice, however, retinal cell loss occurs at approximately one year of life in these mice⁵. Thus, our analysis of these animals will require further investigation at later time points. Correcting the molecular defect in the 216AA mice will not only provide a potential therapy for individuals with this particular mutation, but could also help advance the development of therapies for additional disease mutations that involve pre-mRNA splicing. Notably, more than 50% of the genes associated with deafness are caused by mutations that alter pre-mRNA splicing.

### Methods Summary

### Cell Culture

A plasmid expressing a minigene of human USH1C 216A exons 2-4 and 2′MOEs were transfected into HeLa cells using Lipofectamine 2000 (Invitrogen). Forty-eight hours after transfection, RNA was isolated and analyzed by RT-PCR with primers to plasmid sequences flanking exon 2 and exon 4.

Mice. Ush1c.216A knock-in mice were obtained from Louisiana State University Health Science Center (LSUHSC)³ and bred and treated at Rosalind Franklin University of Medicine and Science (RFUMS). For ABR analysis, mice were shipped 1-2 weeks post-treatment to LSUHSC. All procedures met the NIH guidelines for the care and use of laboratory animals and were approved by the Institutional Animal Care and Use Committees at RFUMS and LSUHSC. Mice were genotyped using ear punch tissue and PCR as described previously⁵. For studies in adult mice, homozygous Ush1c.216AA mice (2-4 months of age) were injected intraperitoneally twice a week for two weeks. RNA was isolated from different tissues using Trizol reagent (Invitrogen) and analyzed by radioactive RT-PCR using primers musUSH1Cex2F and musUSH1Cex5F of the Ush1c.216A transgene. Products were separated on a 6% non-denaturing polyacrylamide gel and quantitated using a Typhoon 9400 phosphorimager (GE Healthsciences). For studies in neonates mice, pups were injected with 300 mg/kg of 2′MOE ASOs at P3-P5 days of age by intraperitoneal injection. After ABR analysis, animals were euthanized and tissues were collected.

mRNA Splicing and protein analysis. Inner ears were isolated, cochleae and vestibules separated and immediately frozen in liquid nitrogen or stored in Trizol reagent. For western blot analysis, proteins were obtained from homogenization in a modified RIPA buffer¹⁰ or isolated from Trizol reagent (Invitrogen) according to manufacturer's instructions. Proteins were separated on 4-15% Tris-glycine gradient gels, transferred to membrane and probed with USH1C (Novus Biologicals) or β-actin (Sigma Aldrich) specific antibodies. RNA was isolated from different tissues using Trizol reagent (Invitrogen) and analyzed by radioactive RT-PCR using primers musUSH1Cex2F and musUSH1Cex5F of the Ush1c.216A transgene. Products were separated on a 6% non-denaturing polyacrylamide gel and quantitated using a Typhoon 9400 phosphorimager (GE Healthsciences).

Behavioral analysis. Mice were placed in an open-field chamber and behavior was analyzed using Anymaze software.

Auditory-Evoked Brain Stem Response

Hearing thresholds of treated and untreated Ush1c wt, het and 216AA mutant mice were measured by auditory-evoked brain stem response (ABR). Mice were anesthetized ((I.P. ketamine, 100 mg/kg; xylacine, 6 mg/kg) and body temperature was maintained near 38° C. with a heat pad. All recordings were conducted in a sound proof room. Stimuli consisted of 5 ms pulses of broad-band, 8-, 16- and 32 kHz, with 0.5 ms linear ramps. The stimuli were broadcast through a Motorola piezoelectric speaker (Model No. 15D87141E02) fitted with a plastic funnel and 2 mm diameter tubing over the speaker front, producing an acoustic wave guide which was positioned in the external meatus approximately 0.5 cm from the tympanum. Using continuous tones, stimulus amplitude was calibrated at the end of the tubing with a Bruel and Kjaer 2610 measuring amplifier (fast, linear weighting), 4135 microphone (grid on) and 4230 pistonphone calibrator. All stimulus amplitudes were dB (SPL; rel 20 μPa). Total harmonic distortion was −40 dB (Hewlet Packard 3562A Signal Analyzer). Stimuli were generated (195 kHz srate) and responses digitized (97.7 kHz srate) using TDT System III hardware and software (Brainware). ABRs were recorded with a silver wire (0.03 o.d.) placed subcutaneously behind the left ear, with indifferent and ground electrodes (steel wire) placed subcutaneously at the vertex and hind-limbs, respectively. Responses to 5 msec broad-band, 8-, 16-, and 32-kHz tone bursts were recorded. After amplification (60 dB, Grass P5 AC), filtering (0.3Hz-1 kHz; TDT PF1), and averaging (n=124-1024), thresholds (+/−6 dB) were determined by eye as the minimum stimulus amplitude which produced an ABR wave pattern similar to that produced for the highest intensity stimulus (90 dB).

Immunofluorescence

Fluorescent labeling of microdissected whole-mount preparations of the organ of Corti were used to study the cochleas of one month old treated and untreated mutant and control mice as described previously¹³. Briefly, cochleae were isolated from the auditory bulla and a small opening was created in the apex. The stapes was removed from the oval window and the cochleae were gently perfused with 4% paraformaldehyde in 0.1M phosphate buffer, pH 7.4 and post-fixed by immersion for 2 hours in the same fixative at 4° C. Segments (half turns) of the organ of Corti were carefully dissected free from the cochlea, the stria vascularis was pulled off or trimmed down, and the tectorial membrane was lifted free with fine forceps and discarded. Tissues were washed twice with PBS following fixation and processed for immunohistochemistry. Tissues were incubated for 1 hour at room temperature in a blocking solution consisting of 10% normal goat serum/0.03% saponin10.1% Triton X-100 in PBS in order to reduce non-specific binding of primary and secondary antibodies. Primary antibody incubations were then performed at 4° C. in PBS containing 0.03% saponin, 3% normal goat serum, 2 mg/ml bovine serum albumin, and 0.1% Triton x-100. A mouse monoclonal anti-parvalbumin antibody (parv19, Cat. No. P3088, Sigma, St. Louis Mo., 1:500; Sage et al., 2000) was used to label cochlear hair cells. A mouse monoclonal anti-neurofilament 200 kDa antibody (Cat. No. N0142, Sigma) was used at a dilution of 1:500 to label nerve fibers (Hardie et al., 2004). A rabbit anti-harmonin antibody (Ush1c, Cat. No., Novus) was used at to label all isoforms of harmonin. To detect the presence of Ush-2′MOE, and anti-Ush-2′MOE antibody (Isis Pharmaceuticals) was used. Secondary antibodies conjugated to Alexa 488, 568 or 633 (Invitrogen/Molecular Probes) were used at a dilution of 1:200 in the same buffer for 2-4 hours at room temperature. For mouse antibodies against parvalbumin, the M.O.M. kit was used as specified by the manufacturer (Vector Labs). Tissues were washed (3 times for 10-15 min. each) after primary and secondary antibody incubations in 0.1% Tween-20 in PBS. After counterstaining nuclei with DAPI (Cat. No. D9542, Sigma-Aldrich, 1 microgram/ml) or Sytox Green specimens were mounted in Fluoromount-G™ (Cat. #0100-01, Southern Biotech, Birmingham Ala.), coverslipped, and examined by confocal fluorescence microscopy. Preparations were examined with an Zeis laser scanning confocal microscopic equipped with 405 nm blue diode multiline argon laser (457 nm, 488 nm and 514 nm), 543 nm helium neon laser, and 637 nm helium neon lasers. Sequential image acquisition was performed when bleed-through between channels was an issue. Files were imported into Image J and/or Adobe Photoshop for processing and analysis.

### REFERENCES

1 Morton, C. C. & Nance, W. E. Newborn hearing screening—a silent revolution. N Engl J Med 354, 2151-2164, doi:354/20/2151 [pii]
10.1056/NEJMra050700 (2006).
2 Kral, A. & O'Donoghue, G. M. Profound deafness in childhood. N Engl J Med 363, 1438-1450, doi:10.1056/NEJMra0911225 (2010).
3 Lentz, J., Pan, F., Ng, S. S., Deininger, P. & Keats, B. Ush1c216A knock-in mouse survives Katrina. Mutat Res 616, 139-144, doi:S0027-5107(06)00320-4 [pii]
10.1016/j.mrfmmm.2006.11.006 (2007).
4 El-Amraoui, A. & Petit, C. Usher I syndrome: unravelling the mechanisms that underlie the cohesion of the growing hair bundle in inner ear sensory cells. J Cell Sci 118, 4593-4603, doi:118/20/4593 [pii]
10.1242/jcs.02636 (2005).
5 Lentz, J. J. et al. Deafness and retinal degeneration in a novel USH1C knock-in mouse model. Dev Neurobiol 70, 253-267, doi:10.1002/dneu.20771 (2010).
6 van Ommen, G. J., van Deutekom, J. & Aartsma-Rus, A. The therapeutic potential of antisense-mediated exon skipping. Curr Opin Mol Ther 10, 140-149 (2008).
7 Hua, Y. et al. Antisense correction of SMN2 splicing in the CNS rescues necrosis in a type III SMA mouse model. Genes Dev 24, 1634-1644, doi:gad.1941310 [pii]
10.1101/gad.1941310 (2010).
8 Goemans, N. M. et al. Systemic administration of PRO051 in Duchenne's muscular dystrophy. N Engl J Med 364, 1513-1522, doi:10.1056/NEJMoa1011367 (2011).
9 Hastings, M. L., Allemand, E., Duelli, D. M., Myers, M. P. & Krainer, A. R. Control of pre-mRNA splicing by the general splicing factors PUF60 and U2AF(65). PLoS One 2, e538, doi:10.1371/journal.pone.0000538 (2007).
10 Hastings, M. L. et al. Tetracyclines that promote SMN2 exon 7 splicing as therapeutics for spinal muscular atrophy. Sci Transl Med 1, 5ra12, doi:10.1126/scitranslmed.3000208 (2009).

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims

We claim:

1. A method for treating Usher's syndrome in a human subject comprising:
administering to the human subject an oligonucleotide having 8 to 30 linked nucleosides having a nucleobase sequence comprising a complementary region comprising at least 8 contiguous nucleobases complementary to a target region of equal length within exon 3 of an Usher transcript.

2. The method of claim 1 wherein the oligonucleotide is chemically modified to be different from the naturally occurring nucleotide.

3. The method of claim 2 wherein the naturally occurring nucleotide comprises a sugar moiety, a base moiety and a phosphodiester linking group and the chemical modified nucleotide has a different sugar moiety, a different base moiety, a different linking group or combinations of any of these modifications.

4. The method of claim 3 wherein the chemical modification is to the sugar moiety.

5. The method of claim 4 wherein the ribose sugar of the naturally occurring nucleoside is replaced by a morpholine ring.

6. The method of claim 4 wherein the ribose sugar of the naturally occurring nucleoside is replaced by a furanosyl.

7. The method of claim 6 wherein the furanosyl has chemical substituents to form bicyclic or tricyclic sugars.
